SEQUENCE LISTING (28 sequences)

SEQ ID NO: 1 - DNA, 25 bp, Artificial Sequence
  sgRNA
caccgcgaagcccatcgaattctac

SEQ ID NO: 2 - DNA, 25 bp, Artificial Sequence
  sgRNA
aaacgtagaattcgatgggcttcgc

SEQ ID NO: 3 - DNA, 25 bp, Artificial Sequence
  sgRNA
caccgtagaattcgatgggcttcgc

SEQ ID NO: 4 - DNA, 25 bp, Artificial Sequence
  sgRNA
aaacgcgaagcccatcgaattctac

SEQ ID NO: 5 - DNA, 25 bp, Artificial Sequence
  sgRNA
caccgcgggcaacacttgcccccta

SEQ ID NO: 6 - DNA, 25 bp, Artificial Sequence
  sgRNA
aaactagggggcaagtgttgcccgc

SEQ ID NO: 7 - DNA, 25 bp, Artificial Sequence
  sgRNA
caccgccgtatgtgatgccagtgta

SEQ ID NO: 8 - DNA, 25 bp, Artificial Sequence
  sgRNA
aaactacactggcatcacatacggc

SEQ ID NO: 9 - DNA, 25 bp, Artificial Sequence
  sgRNA
caccggctgcaacggctgccctttt

SEQ ID NO: 10 - DNA, 25 bp, Artificial Sequence
  sgRNA
aaacaaaagggcagccgttgcagcc

SEQ ID NO: 11 - DNA, 25 bp, Artificial Sequence
  sgRNA
caccggcagcaaccagttcatctcg

SEQ ID NO: 12 - DNA, 25 bp, Artificial Sequence
  sgRNA
aaaccgagatgaactggttgctgcc

SEQ ID NO: 13 - DNA, 25 bp, Artificial Sequence
  sgRNA
caccgggcaggcttaaaggctaacc

SEQ ID NO: 14 - DNA, 25 bp, Artificial Sequence
  sgRNA
aaacggttagcctttaagcctgccc

SEQ ID NO: 15 - DNA, 25 bp, Artificial Sequence
  sgRNA
caccggtcctgcaggggaattgaac

SEQ ID NO: 16 - DNA, 25 bp, Artificial Sequence
  sgRNA
aaacgttcaattcccctgcaggacc

SEQ ID NO: 17 - DNA, 8897 bp, Artificial Sequence
  plasmid
ctttcctgcgttatcccctgattctgtggataaccgtattaccgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggctccaccatgggaaccgacattgattattgactaggcttttgcaaaaagcttggtaccgagctcggatccactagtccagtgtggtggaattctgcagatcatgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgctcgagtctagagggcccttcgaaggtaagcctatccctaaccctctcctcggtctcgattctacgcgtaccggtcatcatcaccatcaccattgagtttatctagacccagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctggtgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 18 - DNA, 8858 bp, Artificial Sequence
  plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacgtttaaacggtctagcagggaggtggccttgttgtgtgtggtggtttgaatattcttggctcagggagtggcactatgaaggggtgtggacttggagtagatgtgtcaatgtaggcgtaggctttaaaacctcatcctagctgcctggaagtcagtattctgctagcagccttcagatgaagatgtagaactctcagctcttccatgcctgtctggatgctgccaagcttcccaccatgatgacaacagactgaacctctgaactgtaagccagccccaatgaaatgttgtcctttataagagttgccttggtcatggtgtctgttcacagcagtacaaccctgactaagacactgggccatcagaggtagcaagctgggaggcattttggaaggctttacataaatgagtatatggcgacattctgcccagaagatccaagcccagagtgcagcctgagtaagaggggagacggctactgtgagcaggtcctgctgggtcggttccctgtgtggagctgccatgttcctcctctcttcccccagacttcacagtgttttcagaatccagtgtccctaagcatttctggccccatctcaagccctcactacaccggcaaaaatgcctgaagtgggagccccaagtactgtagctgcctcatctggctggggaatggtgggccttagccccaagcctccagtacaaagagtaagccccctggctctttctccacacctctgtccctatccttagtcaaaggctgtgccttcctaccaaggttgcctgagcctgtgagcccatgtcagcagtaccctggctctcaccctgccatagtgtaggaggagttgagagttctacatcttcatctgaaggctgctagcagaataaatttttttttttttttttttttttggtgcctggagcagagcaaagatgaagagatgggcaagaccattccacttacaatgtccaagtattacctttctccagctcatcccaagcccttcaaccatgactccacccaactggaagcagaggcaagaggtcccactatgtatcatttcctctgtggcactacctgcccattctctgcaggtccccaggagaggtgcagtgatctgcatcaactatcctctgagaagtccctgcatttcttgcacccatctctcaccactacatgttccttcaccaccacccccctccccccgtttcccctttcctcctttctgtcatccaaggaatatttgctaagggactcctgccagctaggactgagcttttgaggacaggccccagatgcgggtctccaaactgctgtacaggtttagatcaggaatgcaggtcgctaacacttctggctgcaggcgcttaagtaggggttggcaggccagctgagtgtttcaccagcgaagcccgggtgttgcctggctctggtgatccggcaggggtgggtgataactgtgttttttgggaagaacatctgctagaaggaaccatctgagagagcatacctgaacaccctagagtcctcttgagactggccagggtcccttatactgtggaacaaccttcttctcccaactaggttgtgagcttctcgagggcagggctggacttcaaatagccctgtagtctgctccaaagagctgtaggcagagtcatcttgggtgggaacgaacaaatacttggcaaaaagagaaagcggagacctgggcatcccgctggccatgcccctagaaatccactagagacgggttacttggaactgtgttgctactttctgacaaaggggtcagaaacagagctgcaagctatcacgtgggctgtgatcaggtcacgtggtctgaaggtctgcacgtgaagcagggagtcaaacacgagaaggggcggggcaagcccagcacgaagtgggcaggtggggcggggaggcggggggggggggcgtggagtgagacgcttgcataagagtgggcacccaggtgctggttggatgcagagctcctggggtaggcacagtgtgggtgagagggcgggctggtgggcctagctaagggctatctgcggcttccggcttgttgcttccctccaacttcgtgccaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaagcttggcagtggagagggcagaggaagtctgctaacatgcggtgacgtcgaggagaatcctggcccaatggcgtccggtggccacgagcgggccaatgaggattacagagtctctggcattacgggatgcagcaagactcctcagcctgagactcaggacagcttgcagacctcatcacaaagctcagctctctgcacagctcctgtggctgctgcaaacttgggccccagtcttcggagaaacgtggtcagcgagagagaacgcaggaggcggatctcgttgagctgtgagcacttgcgggctctactgcctcagtttgatggccgacgggaggacatggcatctgtcctggagatgtctgtgtacttcctccagcttgcccacagcatggaccctagctgggagcaactctctgttcctcagcctccccaggagatgtggcacatgtggcagggtgatgttctgcaggtaaccctggcgaatcagattgcagacagcaagccagactccggtatagccaaaccatctgctgtgtctcgggtacaggatcccccatgctttgggatgctggatacagaccagagccaggctactgagagagagtcagagctgctggagagaccttcctcctgccctggtcatcgccagagcgcgttgtcattcagtgagccagagtcttccagcttgggtcctgggctcccaccctggatccctcactcatggcagccagccactcccgaggcaagtgacattgttcctggtgggtcacaccaggtggcatccctggccggggaccctgaatcttccggcatgctggctgaggaggccaacttggtcttggcatctgtgcctgatgccaggtacaccacaggggcagggtccgatgtggtggatggagcaccctttctgatgaccaccaatcctgactggtggttggggtcggtggagggcagaggaggcccagcccttgccaggagcagcccagtggatggggcagagccaagcttcatcggagaccctgagctttgctcccaggagctccaggctggtcctggagagctgtggggtttggattttggcagccctggcctggccctgaaggatgaagcggacagcatcttccctgactttttcccctgataccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggatatccttgtttaagggtgccatgccacttgtcttcttgggctccagccttcagcccactctgcacctcatgatacattcagaccataccagttggtattctccttagggggcaagtgttgcccgtatgtgatgccagtgtatgggagtttcagggtctccatatgtccatttctggccaagagcactcacactcagacgtctaccctttgagcctaagactactgctctagtctcagtcccatgtacacttgtgcgtgggtggatacagcctgacccaagagcgggaggttgggggtgggtgtttgaggagtgaagcttcagtccagacacgtctttccctgtgtgaccctgagtttctggctagtttgtgtgtgtttaaccttcaggttcctcagcctccccaggagatgtggcacatgtggcagggtgatgttctgcaggtaaccctggcgaatcagattgcagacagcaagccagactccggtatagccaaaccatctgctgtgtctcggttagtctccctggggccatctttgcgccctgggctctgagcagtagcattagctgtgtaatagtgtcacacacctgtgacatcactgcttgtgctcaaatgcctttagatggtcccttgagagcctgctgtgacacaacattacacagggtgcccttgtcagcagacttggggcactgggaagataggggagggttacttggttcttctcctgctctctggggctctgaactcctcaatgtcttatactttttcagggtacaggatcccccatgctttgggatgctggatacagaccagagccaggctactgagagagagtcagagctgctggagagaccttcctcctgccctggtcatcgccagagcgcgttgtcattcagtgagccaggtaacctatggccctgaaatgaccatcgctgtggctgaaagggctacctcccccatgtccagagtctagaacagctgactgtctcgcctctgcctctgctgaaatcggggcagttgtgaggcctgcagcttttctagctgttttctagtgcacggggtcttgttctccagtgccaagccctggtcaggaggtaccagtgagtcaccaggtgttaattaaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 19 - DNA, 8699 bp, Artificial Sequence
  plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacgtttaaacggtctagcagggaggtggccttgttgtgtgtggtggtttgaatattcttggctcagggagtggcactatgaaggggtgtggacttggagtagatgtgtcaatgtaggcgtaggctttaaaacctcatcctagctgcctggaagtcagtattctgctagcagccttcagatgaagatgtagaactctcagctcttccatgcctgtctggatgctgccaagcttcccaccatgatgacaacagactgaacctctgaactgtaagccagccccaatgaaatgttgtcctttataagagttgccttggtcatggtgtctgttcacagcagtacaaccctgactaagacactgggccatcagaggtagcaagctgggaggcattttggaaggctttacataaatgagtatatggcgacattctgcccagaagatccaagcccagagtgcagcctgagtaagaggggagacggctactgtgagcaggtcctgctgggtcggttccctgtgtggagctgccatgttcctcctctcttcccccagacttcacagtgttttcagaatccagtgtccctaagcatttctggccccatctcaagccctcactacaccggcaaaaatgcctgaagtgggagccccaagtactgtagctgcctcatctggctggggaatggtgggccttagccccaagcctccagtacaaagagtaagccccctggctctttctccacacctctgtccctatccttagtcaaaggctgtgccttcctaccaaggttgcctgagcctgtgagcccatgtcagcagtaccctggctctcaccctgccatagtgtaggaggagttgagagttctacatcttcatctgaaggctgctagcagaataaatttttttttttttttttttttttggtgcctggagcagagcaaagatgaagagatgggcaagaccattccacttacaatgtccaagtattacctttctccagctcatcccaagcccttcaaccatgactccacccaactggaagcagaggcaagaggtcccactatgtatcatttcctctgtggcactacctgcccattctctgcaggtccccaggagaggtgcagtgatctgcatcaactatcctctgagaagtccctgcatttcttgcacccatctctcaccactacatgttccttcaccaccacccccctccccccgtttcccctttcctcctttctgtcatccaaggaatatttgctaagggactcctgccagctaggactgagcttttgaggacaggccccagatgcgggtctccaaactgctgtacaggtttagatcaggaatgcaggtcgctaacacttctggctgcaggcgcttaagtaggggttggcaggccagctgagtgtttcaccagcgaagcccgggtgttgcctggctctggtgatccggcaggggtgggtgataactgtgttttttgggaagaacatctgctagaaggaaccatctgagagagcatacctgaacaccctagagtcctcttgagactggccagggtcccttatactgtggaacaaccttcttctcccaactaggttgtgagcttctcgagggcagggctggacttcaaatagccctgtagtctgctccaaagagctgtaggcagagtcatcttgggtgggaacgaacaaatacttggcaaaaagagaaagcggagacctgggcatcccgctggccatgcccctagaaatccactagagacgggttacttggaactgtgttgctactttctgacaaaggggtcagaaacagagctgcaagctatcacgtgggctgtgatcaggtcacgtggtctgaaggtctgcacgtgaagcagggagtcaaacacgagaaggggcggggcaagcccagcacgaagtgggcaggtggggcggggaggcggggggggggggcgtggagtgagacgcttgcataagagtgggcacccaggtgctggttggatgcagagctcctggggtaggcacagtgtgggtgagagggcgggctggtgggcctagctaagggctatctgcggcttccggcttgttgcttccctccaacttcgtgccaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaagcttggcagtggagagggcagaggaagtctgctaacatgcggtgacgtcgaggagaatcctggcccaatggcgtccggtggccacgagcgggccaatgaggattacagagtctctggcattacgggatgcagcaagactcctcagcctgagactcaggacagcttgcagacctcatcacaaagctcagctctctgcacagctcctgtggctgctgcaaacttgggccccagtcttcggagaaacgtggtcagcgagagagaacgcaggaggcggatctcgttgagctgtgagcacttgcgggctctactgcctcagtttgatggccgacgggaggacatggcatctgtcctggagatgtctgtgtacttcctccagcttgcccacagcatggaccctagctgggagcaactctctgttcctcagcctccccaggagatgtggcacatgtggcagggtgatgttctgcaggtaaccctggcgaatcagattgcagacagcaagccagactccggtatagccaaaccatctgctgtgtctcgggtacaggatcccccatgctttgggatgctggatacagaccagagccaggctactgagagagagtcagagctgctggagagaccttcctcctgccctggtcatcgccagagcgcgttgtcattcagtgagccagagtcttccagcttgggtcctgggctcccaccctggatccctcactcatggcagccagccactcccgaggcaagtgacattgttcctggtgggtcacaccaggtggcatccctggccggggaccctgaatcttccggcatgctggctgaggaggccaacttggtcttggcatctgtgcctgatgccaggtacaccacaggggcagggtccgatgtggtggatggagcaccctttctgatgaccaccaatcctgactggtggttggggtcggtggagggcagaggaggcccagcccttgccaggagcagcccagtggatggggcagagccaagcttcatcggagaccctgagctttgctcccaggagctccaggctggtcctggagagctgtggggtttggattttggcagccctggcctggccctgaaggatgaagcggacagcatcttccctgactttttcccctgataccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactccatatgtccatttctggccaagagcactcacactcagacgtctaccctttgagcctaagactactgctctagtctcagtcccatgtacacttgtgcgtgggtggatacagcctgacccaagagcgggaggttgggggtgggtgtttgaggagtgaagcttcagtccagacacgtctttccctgtgtgaccctgagtttctggctagtttgtgtgtgtttaaccttcaggttcctcagcctccccaggagatgtggcacatgtggcagggtgatgttctgcaggtaaccctggcgaatcagattgcagacagcaagccagactccggtatagccaaaccatctgctgtgtctcggttagtctccctggggccatctttgcgccctgggctctgagcagtagcattagctgtgtaatagtgtcacacacctgtgacatcactgcttgtgctcaaatgcctttagatggtcccttgagagcctgctgtgacacaacattacacagggtgcccttgtcagcagacttggggcactgggaagataggggagggttacttggttcttctcctgctctctggggctctgaactcctcaatgtcttatactttttcagggtacaggatcccccatgctttgggatgctggatacagaccagagccaggctactgagagagagtcagagctgctggagagaccttcctcctgccctggtcatcgccagagcgcgttgtcattcagtgagccaggtaacctatggccctgaaatgaccatcgctgtggctgaaagggctacctcccccatgtccagagtctagaacagctgactgtctcgcctctgcctctgctgaaatcggggcagttgtgaggcctgcagcttttctagctgttttctagtgcacggggtcttgttctccagtgccaagccctggtcaggaggtaccagtgagtcaccaggtgttaattaaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 20 - DNA, 9539 bp, Artificial Sequence
  plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacgtttaaacccaaggcggaggatcaatataccgtaaaggccaaacgggagtggcttttgacgagaagcagagacggggagctttttaatggatagcaagtgctccggtcttgtcaggtggtttcgttcatcttgtatatgtatgagtgtgtatgtgtgtgtgttatgacatcagagcttcaagtgtgtgtgttatcgaactcgggaccccacatctgcagcccaggttttagaactagcaagaagaaacagacatttgaacatacttttttttttctttctggtttttcgagacagggtttctctgtatagccctggctgtcctggaactcacaccaggctggcctcgaactcagaaatccacctgcctctgcctcccaagtgctgcgccacttcttagagatgctttgaggcaatatctttggaacgagaagtttgtgggcagcggaagttggtcatttaaatgatggggggtggtggtgggttatggacggcgggaagctcagcgccaattcagtgactgttagcggaaaggcgtgcaagagcgggaaagaaggcgccaaaaaaaccgcatccaagaacgaggggccgatgggatgcgcacgagagtttttgaacattgaatcagaccaatcacagagcgtgactcattctgtctgcacgacgagggcggggcactgagtgacagacagaccaatcacagagcgtgactctctggtaaagcgtgaatctctccttccggcctcggcttcgggccaccagagacggagagacggagacggagtcggagacgcgagacgccagcaagcgtttcgggtgtgggagagcagacgcctccctgtttaacaactttctcctggatttgcagcttcctcaacgtccctgcaccttcaggtaataagtaatgggtatagttttctggcagaagtgtctttttgagaaattagagacttgaggacagaatattaaaatatattcgtgttccataggctggagccagacatttaaaaaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaagcttggcagtggagagggcagaggaagtctgctaacatgcggtgacgtcgaggagaatcctggcccaatggaccgcattactgacttttacttcttggacttcagagaatctgttaaaaccctgatcataactggtaattcatggagactacaagaaatgattgacagattcttcacaaacatatcaaatttcaacagagagtctctgactgaaatacagaatattcagattgaagaaattgcagtgaacctgtggaactgggcagttactaagagagtagaactgtctgtgaggaaaaaccaggcagctaaactgtgttatattgcttgcaagctggtatatatgcatggaatctcagtctcttcagaagaagctattcaaagacagattttgatgaatataaaaacaggaaaagagtggttgtatactggaaatgctcagattgctgatgaattttttcaagctgccatgactgatctggagagattatatgtcagattaatgcagagctgctacaccgaggccaacgtgtgtgtgtataagatgattgttgagaaaggcatcttccatgtgctttcttaccaagctgagtcagctgttgctcaaggggatttcaagaaagcatctatgtgcgtcttacgttgcaaagatatgctgatgagactccctaacatgacaaaatatcttcatgtactctgttacaaccttggcatagaagcaagcaagcggaataaatacaaagagagttcattctggcttggccaaagctatgaaattgggaagatggataggcgttctgttgagccacaaatgctggctaaaacgctgcggttactagccactatttatttgaattgtggtggcgaagcatattataccaaggccttcattgctatactcattgcaaacaaggaacatttacatccagctgggcttttcttaaagatgaggatcctcatgaaaggcaactcatgtaatgaagaactccttgaagctgctaaggaaatactatatcttgctatgcctttggaattctatctgagcattattcaattcctgatagataataaaagagagtctgttgggtttcgctttctgagaatcatctctgacaattttaagtcgccagaagataggaagagaattctgttgttctacattgacacgcttttacaaaaggatcaagacatgattgctgaagagaagattaaagacgtccttaaaggttaccaaacaagaagtcgactgtcaagagatttggtaaattggttacacaacattctgtggggaaaggcttccagaagtgttaaggtccaaaaatatgctgatgccctacactggtacagttattctctgaagttgtatgagtatgataaagcagatctggatttgatcaagctgaagaggaacatggtttcctgttacttatctttgaaacaacttgataaggctaaagaggccatagcagaagttgagcaaaaggatcctacacatgttttcactcggtattatatattcaagatcgcaatcatggagggtgatgctttcagagctttacaggtggtcagtgctttaaagaaatcattaatggatggagaatcagaagatcgtggactaattgaagctggagtttcaactctcacaatcctaagtttatctatagattttgctctagagaatggacagcaatttgtggcagaaagagctttggaatatttatgtcaactttcaaaagacccaaaagaagtacttggaggtttaaagtgtctcatgcggattattcttccacaagcttttcatatgccagaatctgaatataaaaagaaagaaatgggtagactttggaactacttgaatacagcactcctgaaattttctgaatattttaatgaagctccctcaactttggattatatggttaatgatgccaattggttcaggaaaatagcttggaacttagctgtgcaatctgagaaggatctagaggcaatgaaaaactttttcatggtttcttataagctgtcccttttttgtcctttggatcaaggactactgattgcacagaaaacgtgtttacttgtagcagctgcagttgatctggatagaggaagaaaagctccaacaatttgtgagcagaacatgttactaagaacagcacttgagcagataaagaaatgcaaaaaagtttggaatctcctgaaaaaaacaggggacttctcaggtgatgactgtggggtattgcttctgctctatgaatttgaagttaaaaccaaaacgaatgatccatcactgagcagatttgtggattcagtttggaagatgcctgatttagaatgcagaacacttgaaacaatggcattactagctatggataaacctgcatactatcctactattgcacataaggccatgaaaaaacttttattgatgtacagaaaacaggagccagttgatgttttaaaatacagcgtatgcatgcacaacttgattaaacttctggtggcagatgaagtatggaatatatcgctgtatcccctaaaagaagttcagagccattttaaaaatactctgagcatcattcgccaaaacgaaggatacccagaagaggagattgtatggctaatgatcaagtcttggaatattggaatactgatgtctagcaagaacaagtatatatctgcagaaaggtgggctgcaatggcattggatttccttggccaccttagcaccctcaaaacaagctatgaagcaaaggtgaatcttctgtatgccaacctcatggaaatattagataaaaagacggatttaagatctacagagatgactgaacaattaagagcacttattgttcctccggaggatcaaggttcagtttccagcaccaacgtggcagctcaaaaccatctgtaataccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaccatgtctgaatctcccatagacaattttaaggtgtctcatccgcccagtctctgcagtacttcgtctcttagccttggcactgcagttagacttttgcttgcgcttggcagggtagccacgtttgccacagatagatttctgatagtggtcgtccttagagccacggagaccggcggtacaatgtgtgcctgtttttgggatgctttccaaaggatgccgttccttttgtcatgttgcttctacctccgaggccaaagagactggaagagcccctttttggcttttttttttttttcaagacagggtttctctgtgtagccctggctgtcctggaactcactctgtagaccaggctggcctcgaactcagaaatccgcctgcctctgccaccaccgcctggcttcctctttggcttcttgagacagagtttttactctgtgtctcaggctgacctagaactttatgtagcccaggctatcctcaaacttgcaatcttcctacctaagcttccagagtgcaggtgtcaaacaccacacctggcctataatcccagcatttgtaagactaaggctggagcaacacctcaaatattgtttcccaaaacaaaaccaaacaaaatgaaaacaagctcggagagagctctgtggcagtgtttgccttccacatgtaaatccctggagtggaggcccagcagtgcagtttgtctcataagtttgtcttttcacagtactcagtgcaccagttttcctcatgtatgtaccagaaataatatttggaaccttttatgaactagtgtaaggaaacaaaattgacaatccatagcttattttctggtcataatctatagcttttccccaggaattttaggagatgcggaatcatactgtttaacctgaaaaaatactgtgtaaaaagtatatttaagctgggcagtggtggcgcatccctgtaatcccagcacttgggaggcagaggcaggtggatttctgagttcgaggccagcctgatctacagagtgagttccagtacagccagggctacacagagaaaccctgtctcgaaaaaccaaaaaaaaaaaaaaaaaaaaaaaaggacattcaggaagttgttgcaagcttaattaaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 21 - DNA, 7630 bp, Artificial Sequence
  plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacgtttaaaccttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctagaagatgggcgggagtcttgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgagggcagaggaagtctgctaacatgcggtgacgtcgaggagaatcctggcccaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaagcttggcagtggagagggcagaggaagtctgctaacatgcggtgacgtcgaggagaatcctggcccaatggcgtccggtggccacgagcgggccaatgaggattacagagtctctggcattacgggatgcagcaagactcctcagcctgagactcaggacagcttgcagacctcatcacaaagctcagctctctgcacagctcctgtggctgctgcaaacttgggccccagtcttcggagaaacgtggtcagcgagagagaacgcaggaggcggatctcgttgagctgtgagcacttgcgggctctactgcctcagtttgatggccgacgggaggacatggcatctgtcctggagatgtctgtgtacttcctccagcttgcccacagcatggaccctagctgggagcaactctctgttcctcagcctccccaggagatgtggcacatgtggcagggtgatgttctgcaggtaaccctggcgaatcagattgcagacagcaagccagactccggtatagccaaaccatctgctgtgtctcgggtacaggatcccccatgctttgggatgctggatacagaccagagccaggctactgagagagagtcagagctgctggagagaccttcctcctgccctggtcatcgccagagcgcgttgtcattcagtgagccagagtcttccagcttgggtcctgggctcccaccctggatccctcactcatggcagccagccactcccgaggcaagtgacattgttcctggtgggtcacaccaggtggcatccctggccggggaccctgaatcttccggcatgctggctgaggaggccaacttggtcttggcatctgtgcctgatgccaggtacaccacaggggcagggtccgatgtggtggatggagcaccctttctgatgaccaccaatcctgactggtggttggggtcggtggagggcagaggaggcccagcccttgccaggagcagcccagtggatggggcagagccaagcttcatcggagaccctgagctttgctcccaggagctccaggctggtcctggagagctgtggggtttggattttggcagccctggcctggccctgaaggatgaagcggacagcatcttccctgactttttcccctgaaacttgtttattgcagcttataatggttacaaataaagcaatagcatcacaaatttcacaaataaagcatttttttcactgcattctagttgtggtttgtccaaactcatcaatgtatcttagtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgagctattaaccattaatggtttccaggtggatgtctcctcccccaatattacctgatgtatcttacatattgccaggctgatattttaagacattaaaaggtatatttcattattgagccacatggtattgattactgcttactaaaattttgtcattgtacacatctgtaaaaggtggttccttttggaatgcattaattaaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 22 - DNA, 9400 bp, Artificial Sequence
  plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacgtttaaaccttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctagaagatgggcgggagtcttgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgagggcagaggaagtctgctaacatgcggtgacgtcgaggagaatcctggcccaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaagcttggcagtggagagggcagaggaagtctgctaacatgcggtgacgtcgaggagaatcctggcccaatggaccgcattactgacttttacttcttggacttcagagaatctgttaaaaccctgatcataactggtaattcatggagactacaagaaatgattgacagattcttcacaaacatatcaaatttcaacagagagtctctgactgaaatacagaatattcagattgaagaaattgcagtgaacctgtggaactgggcagttactaagagagtagaactgtctgtgaggaaaaaccaggcagctaaactgtgttatattgcttgcaagctggtatatatgcatggaatctcagtctcttcagaagaagctattcaaagacagattttgatgaatataaaaacaggaaaagagtggttgtatactggaaatgctcagattgctgatgaattttttcaagctgccatgactgatctggagagattatatgtcagattaatgcagagctgctacaccgaggccaacgtgtgtgtgtataagatgattgttgagaaaggcatcttccatgtgctttcttaccaagctgagtcagctgttgctcaaggggatttcaagaaagcatctatgtgcgtcttacgttgcaaagatatgctgatgagactccctaacatgacaaaatatcttcatgtactctgttacaaccttggcatagaagcaagcaagcggaataaatacaaagagagttcattctggcttggccaaagctatgaaattgggaagatggataggcgttctgttgagccacaaatgctggctaaaacgctgcggttactagccactatttatttgaattgtggtggcgaagcatattataccaaggccttcattgctatactcattgcaaacaaggaacatttacatccagctgggcttttcttaaagatgaggatcctcatgaaaggcaactcatgtaatgaagaactccttgaagctgctaaggaaatactatatcttgctatgcctttggaattctatctgagcattattcaattcctgatagataataaaagagagtctgttgggtttcgctttctgagaatcatctctgacaattttaagtcgccagaagataggaagagaattctgttgttctacattgacacgcttttacaaaaggatcaagacatgattgctgaagagaagattaaagacgtccttaaaggttaccaaacaagaagtcgactgtcaagagatttggtaaattggttacacaacattctgtggggaaaggcttccagaagtgttaaggtccaaaaatatgctgatgccctacactggtacagttattctctgaagttgtatgagtatgataaagcagatctggatttgatcaagctgaagaggaacatggtttcctgttacttatctttgaaacaacttgataaggctaaagaggccatagcagaagttgagcaaaaggatcctacacatgttttcactcggtattatatattcaagatcgcaatcatggagggtgatgctttcagagctttacaggtggtcagtgctttaaagaaatcattaatggatggagaatcagaagatcgtggactaattgaagctggagtttcaactctcacaatcctaagtttatctatagattttgctctagagaatggacagcaatttgtggcagaaagagctttggaatatttatgtcaactttcaaaagacccaaaagaagtacttggaggtttaaagtgtctcatgcggattattcttccacaagcttttcatatgccagaatctgaatataaaaagaaagaaatgggtagactttggaactacttgaatacagcactcctgaaattttctgaatattttaatgaagctccctcaactttggattatatggttaatgatgccaattggttcaggaaaatagcttggaacttagctgtgcaatctgagaaggatctagaggcaatgaaaaactttttcatggtttcttataagctgtcccttttttgtcctttggatcaaggactactgattgcacagaaaacgtgtttacttgtagcagctgcagttgatctggatagaggaagaaaagctccaacaatttgtgagcagaacatgttactaagaacagcacttgagcagataaagaaatgcaaaaaagtttggaatctcctgaaaaaaacaggggacttctcaggtgatgactgtggggtattgcttctgctctatgaatttgaagttaaaaccaaaacgaatgatccatcactgagcagatttgtggattcagtttggaagatgcctgatttagaatgcagaacacttgaaacaatggcattactagctatggataaacctgcatactatcctactattgcacataaggccatgaaaaaacttttattgatgtacagaaaacaggagccagttgatgttttaaaatacagcgtatgcatgcacaacttgattaaacttctggtggcagatgaagtatggaatatatcgctgtatcccctaaaagaagttcagagccattttaaaaatactctgagcatcattcgccaaaacgaaggatacccagaagaggagattgtatggctaatgatcaagtcttggaatattggaatactgatgtctagcaagaacaagtatatatctgcagaaaggtgggctgcaatggcattggatttccttggccaccttagcaccctcaaaacaagctatgaagcaaaggtgaatcttctgtatgccaacctcatggaaatattagataaaaagacggatttaagatctacagagatgactgaacaattaagagcacttattgttcctccggaggatcaaggttcagtttccagcaccaacgtggcagctcaaaaccatctgtaaaacttgtttattgcagcttataatggttacaaataaagcaatagcatcacaaatttcacaaataaagcatttttttcactgcattctagttgtggtttgtccaaactcatcaatgtatcttagtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgagctattaaccattaatggtttccaggtggatgtctcctcccccaatattacctgatgtatcttacatattgccaggctgatattttaagacattaaaaggtatatttcattattgagccacatggtattgattactgcttactaaaattttgtcattgtacacatctgtaaaaggtggttccttttggaatgcattaattaaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 23 - DNA, 20 bp, Artificial Sequence
  sgRNA for SOHLH1
atttcagattcttgcttcct

SEQ ID NO: 24 - DNA, 20 bp, Artificial Sequence
  sgRNA for TEX11
ctgggccagaaatgctggta

SEQ ID NO: 25 - DNA, 25 bp, Artificial Sequence
  top strand
caccgctgggccagaaatgctggta

SEQ ID NO: 26 - DNA, 25 bp, Artificial Sequence
  bottom strand
aaactaccagcatttctggcccagc

SEQ ID NO: 27 - DNA, 25 bp, Artificial Sequence
  top strand
caccgcaacgagtgccacatttcct

SEQ ID NO: 28 - DNA, 25 bp, Artificial Sequence
  bottom strand
aaacaggaaatgtggcactcgttgc

(19) United States
(12) Patent Application Publication
(10) Pub. No.: US 2020/0171171 A1
(43) Pub. Date: Jun. 4, 2020

(54) GENE THERAPY FOR TREATMENT OF INFERTILITY

(71) Applicant: University of Pittsburgh - Of the Commonwealth System of Higher Education, Pittsburgh, PA (US)

(72) Inventors: Kyle E. Orwig, Wexford, PA (US); Chatchanan Doungkamchan, Pittsburgh, PA (US); Alexander N. Yatsenko, Wexford, PA (US); Aleksandar Rajkovic, San Francisco, CA (US)

(73) Assignee: University of Pittsburgh - Of the Commonwealth System of Higher Education, Pittsburgh, PA (US)

(21) Appl. No.: 16/630,589

(22) PCT Filed: Jul. 26, 2018
(86) PCT No.: PCT/US2018/043948

§ 371 Date: Jan. 13, 2020

Related U.S. Application Data
(60) Provisional application No. 62/537,370, filed on Jul. 26, 2017.

(51) Int. Cl. A61K 48/00; C12N 15/85; C12N 9/22; A61B 17/435; A61D 19/04

(52) U.S. Cl. CPC A61K 48/0058; C12N 15/85; C12N 2800/80; A61B 17/435; A61D 19/04; C12N 9/22

(57) ABSTRACT

Provided are ex vivo and in vivo methods utilizing therapeutic genes for treatment of male and female infertility, including non-obstructive azoospermia (NOA) and premature ovarian insufficiency (POI) and comorbid diseases, with or without transmitting the therapeutic gene to offspring of the infertile subject. Germline gene therapy methods are also described to reduce or eliminate disease from families with or without transmission of the therapeutic gene to offspring.

### CROSS-REFERENCE TO RELATED APPLICATION

This application claims priority to U.S. Provisional Application No. 62/537,370, filed Jul. 26, 2017, herein incorporated by reference in its entirety.

### FIELD

Methods (ex vivo and in vivo) for treating male and female infertility, including non-obstructive azoospermia (NOA) or premature ovarian insufficiency (POI), are provided, which can be implemented with or without transmitting the therapeutic gene to the offspring of the infertile subject.

### BACKGROUND

Azoospermia (no sperm in the ejaculate) impacts 1% of men in the general population and 10-15% of infertile men¹⁻³, which translates to 645,000 males between the ages of 20 and 50 (prime reproductive age) in the United States alone. Similarly, premature ovarian insufficiency impacts 1% of women under the age of 40 in the United States.

Azoospermia can be categorized as obstructive (OA) or non-obstructive (NOA). Sperm can be recovered directly from the testes of most men with OA by testicular sperm extraction (TESE) or related sperm retrieval procedures with nearly 100% efficiency. In contrast, sperm recovery rates for men with NOA (85% of cases) are much lower, ranging from 0% and 50%⁴, depending on the phenotype. There are currently no options for men with NOA and failed TESE to have biological children. Similarly, there a few options to help women with POI have biological children.

### SUMMARY

The inventors developed gene therapy methods for males with non-obstructive azoospermia (NOA) and females with premature ovarian insufficiency (POI), among the most intractable of infertility diagnoses. The methods include ex vivo gene therapy, which can be followed by transplantation of male or female germline stem cells. The disclosed methods can be implemented without transmission of the therapeutic gene to the offspring of the infertile patient. Thus, the infertile patient can be treated, and the pathogenic mutation can be eliminated or diluted from his/her entire family lineage without transmission of the gene therapy construct to progeny.

In one example, disclosed methods treat a recessive genetic mutation associated with infertility (e.g., autosomal recessive or sex-chromosome-linked recessive), without transmitting genetic modifications to progeny, while eliminating the infertile phenotype from the family lineage. In addition, the disclosed methods, in addition to treating infertility (e.g., NOA or POI), can also eliminate comorbid genetic diseases from the family lineage without germline transmission.

Methods for treating non-obstructive azoospermia (NOA) in a male subject (e.g., infertile subject), wherein the NOA is caused by a genetic mutation, such as one that causes a germ cell development defect and has a recessive or dominant mode of inheritance. Thus, such mutations may affect development of sperm or sperm precursor cells, such as primordial germ cells, pre-spermatogonia, pro-spermatogonial, gonocytes, spermatogonial stem cells, undifferentiated spermatogonia, differentiated spermatogonia, spermatocytes, and/or spermatids). In one example, such methods can include introducing one or more recombinant nucleic acid molecules into spermatogonial stem cells (SSCs) from the testes of the subject, resulting transformed SSCs, wherein the nucleic acid molecule corrects the genetic mutation causing the NOA (e.g., wherein the nucleic acid molecule corrects at least one allele of the mutation). Transformed SSCs that are heterozygous or hemizygous for the genetic mutation can be isolated or purified, thereby generating isolated transformed SSCs. The isolated transformed SSCs that are heterozygous or hemizygous for the genetic mutation are introduced or transplanted the into the male subject, thereby treating NOA in the subject. In another example, the method includes introducing one or more recombinant nucleic acid molecules into induced pluripotent stem cells (iPSCs) of the male subject, wherein the nucleic acid molecule corrects the genetic mutation causing the NOA, thereby generating transformed iPSCs (e.g., wherein the nucleic acid molecule corrects at least one allele of the mutation). Transformed iPSCs that are heterozygous or hemizygous for the genetic mutation can be isolated or purified, thereby generating isolated transformed iPSCs. The isolated transformed iPSCs that are heterozygous or hemizygous for the genetic mutation are differentiated into primordial germ cell-like cells (PGCLCs), which are then either (1) transplanted or introduced into the testes of the male subject (this regenerates spermatogenesis in vivo, e.g., produces sperm in the testes), or (2) differentiated into sperm in vitro, thereby treating NOA in the subject.

Methods for treating premature ovarian insufficiency (POI) (also known as premature ovarian failure (POF), primary ovarian insufficiency, and primary ovarian failure) in a female subject (e.g., infertile subject), wherein the POI is caused by a genetic mutation, such as one that causes a germ cell development defect and has a recessive or dominant mode of inheritance. Thus, such mutations may affect development of eggs or egg precursor cells, such as primordial germ cells, oogonia or developing oogonia (eggs) in developing follicles including primordial follicles, secondary follicles, tertiary follicles, antral follicles or Graffian follicles. In one example, the method includes introducing one or more recombinant nucleic acid molecules into induced pluripotent stem cells (iPSCs) of the female subject, wherein the nucleic acid molecule corrects the genetic mutation causing the POI, thereby generating transformed iPSCs (e.g., wherein the nucleic acid molecule corrects at least one allele of the mutation). Transformed iPSCs that are heterozygous or hemizygous for the genetic mutation can be isolated or purified, thereby generating isolated transformed iPSCs. The isolated transformed iPSCs that are heterozygous or hemizygous for the genetic mutation are differentiated into primordial germ cell-like cells (PGCLCs), which are then either (1) transplanted or introduced into the ovary of the female subject (this regenerates oogenesis in vivo, e.g., produces eggs in the ovary), or (2) differentiated into eggs in vitro, thereby treating POI in the subject. The resulting in vivo-derived eggs can be collected from the ovaries of the treated female subject; or the in vitro-derived eggs from the treated female subject are fertilized with sperm to produce embryos.

The foregoing and other objects and features of the disclosure will become more apparent from the following detailed description, which proceeds with reference to the accompanying figures.

### BRIEF DESCRIPTION OF THE DRAWINGS

FIGS. 1A-1C. In vivo somatic cell gene therapy. (A) Gene therapy vector is injected into the testicular seminiferous tubules or into the interstitial space, depending on target somatic cell type (e.g., Sertoli cells, peritubular myoid cells and Leydig cells). If the therapy is effective, treated males should be producing sperm within a few weeks of treatment. (B) Treated males can then be bred to fertile females to produce babies the normal way. (C) If sperm counts are low, sperm can be retrieved from the ejaculate or directly from the testis and used to fertilize eggs using in vitro fertilization with intracytoplasmic sperm injection.

FIGS. 2A-2D. SCARKO mice: model of human NOA. (A) SCARKO mice have small testes compared to littermate controls and are infertile (B). Compared with controls (C), SCARKO mice (D) have smaller seminiferous tubules with no lumen and incomplete spermatogenesis.

FIG. 3. Adeno-EF1a-eGFP-AR gene therapy vector. The vector features an EF1a promotor to direct expression of an eGFP reporter gene and a human androgen receptor (hAR) gene.

FIGS. 4A-4L. Expression of the eGFP reporter gene indicates that Ad-EF1a-eGFP-hAR efficiently transduces Sertoli cells along the length of the recipient mouse seminiferous tubules. (A) Whole testes bright field. scale bar: 2 mm. (B) Whole testes dark field viewed under an epifluorescent microscope using a FITC filter. The left testis is uninjected; the right testis is injected with Ad-EF1a-eGFP-hAR. (C) Dissected seminiferous tubules bright field. (D) Dissected seminiferous tubules dark field. (E-H) Higher magnification presentation of uninjected seminiferous tubules in whole mount bright field (E) and dark field (F) and also in section (G and H). Dapi staining in (G) marks all cell nuclei. Scale bar: 100 μm. (I-J) Higher magnification presentation of seminiferous tubules injected with the Ad-EF1a-eGFP-hAR gene therapy vector in whole mount (I-J) and section (K-L).

FIGS. 5A-5I. AD-EF1a-eGFP-hAR restores sperm production in SCARKO mice. The testes of SCARKO mice were injected with Ad-EF1a-eGFP-Empty (A-B) or Ad-EF1a-eGFP-hAR (C-D). Testes were collected three weeks after injection and analyzed histologically. Animals injected with the Ad-EF1a-eGFP-Empty vector maintained the NOA with maturation arrest phenotype and had no tubules with spermatids or sperm (A-B). Animals injected with Ad-EF1a-eGFP-hAR exhibited complete spermatogenesis in over 90% of tubules (C-E). (F-H) Sperm recovered from the epididymis of Ad-EF1a-eGFP-hAR treated SCARKO mice 3 months after injection were competent to fertilize mouse eggs, leading to preimplantation embryo development (2-cell, 4-cell, 8-cell, etc). When the resulting embryos were transferred to pseudopregnant females, they gave rise to normal offspring (I).

FIGS. 6A-6D. Adeno-EF1a-eGFP-hAR transduces Sertoli cells, but not germ cells. Immunofluorescent co-staining for the eGFP reporter and Sox9 (Sertoli cell marker) indicates that Sertoli cells were transduced with the Ad-EF1a-eGFP-hAR adenovirus (A-B). Co-staining for the eGFP reporter and VASA (germ cell marker) reveals no overlap indicating that germ cells were not transduced with the Ad-EF1a-eGFP-hAR adenovirus (C-D).

FIGS. 7A-7B. Immunostaning for ZBTB16 in (A) wild type and (B) Soh1h1−/mice. Soh1h1−/−mice are infertile with a NOA-MA phenotype. Seminiferous tubules contain ZBTB16+ spermatogonia indicated by red staining, but not differentiating germ cells (spermatocytes, spermatids; Suzuki et al., Dev. Biol. 361:301-12, 2012).

FIG. 8. Ex vivo germline gene therapy for autosomal recessive disorder: inserting therapeutic gene into endogenous locus.

FIG. 9. Ex vivo germline gene therapy for autosomal recessive disorder: inserting therapeutic gene into the ROSA (“Safe Harbor”) locus.

FIG. 10. Gene therapy constructs. The genetic elements for CRISPR/Cas9-mediated gene editing include the guide RNA (sgRNA) specific for the mutation of interest, a Cas9 endonuclease, and a donor DNA template specific for the target gene. These elements can be introduced into target cells, for example by transfection, electroporation, viral transduction or other approaches. These genetic elements can be added separately or in various composite combinations. The constructs depicted in this figure are examples independent constructs that can be used for ex vivo gene editing of spermatogonial stem cells (SSCs) from the testis or ex vivo gene editing of male or female patient-derived induced pluripotent stem cells (iPSCs). Construct 1 features a U6 promoter-driven sgRNA. Construct 2 features a CMV promoter-driven bicistronic Cas9-eGFP transgene. Construct 3 is a donor DNA template that features a promoterless Soh1h1 cDNA and a PGK promoter-driven puromycin resistance gene flanked by left and right homology arms. Abbreviations: pA, polyadenylation sequence; LHA, left homology arm; RHA, right homology arm.

FIG. 11. Ex vivo germline gene therapy for an sex chromosome-linked recessive disorder: inserting therapeutic gene into the endogenous locus.

FIGS. 12A-12D. Polyethyleneimine (PEI) efficiently transfects spermatogonial stem cells. (A) SSC cultures were established from the testis cells of EF1a-eGFP mice in which all cells are green. The EF1a-eGFP SSCs were transfected with PEI and a linearized plasmid containing the mCherry reporter gene (red). (B) Flow analysis indicates that almost 70% of transfected cells expressed the mCherry reporter gene. (C-D) Upon transplantation into the testes of infertile recipient mice, transfected SSCs produced colonies of green spermatogenesis that were qualitatively and quantitatively similar to WT SSCs.

FIGS. 13A-13B. Validation of sgRNAs targeting the human SOHLH1 and TEX11 genes. sgRNAs targeting SOHLH1 and TEX11 were designed to target known human mutations identified in NOA patients. The sgRNA targeting SOHLH1 was designed to target the c.346-1G>A mutation, which is located at the splicing acceptor sequence of SOHLH1 intron 3, whereas the sgRNA targeting TEX11 was designed to target the a c.792+1G->A mutation, which is the splicing donor sequence at intron 11. To validate the sgRNAs, 293AD cells were transfected with plasmid DNA containing the sgRNA and Cas9 sequences using PEI. Cells were collected 72 hours later and polymerase chain reaction (PCR) was performed using forward and reverse primers flanking the genomic region targeted region by sgRNAs. The amplicons, which are approximately 1.2 kb for SOHLH1 and 570 bp for TEX11, were then denatured and reannealed to allow mismatch paring between the mutated and the wild-type strands. T7 Endonuclease I (T7E1) was then used to specifically digest the mismatch duplex at the sgRNA-targeted site, giving two smaller fragments of 750 bp (A, top/red arrow) and 462 bp (A, bottom/brown arrow) in SOHLH1 case, and 400-bp (B, red arrow) and 170-bp fragment (B, bottom/brown arrow) in TEX11 case. This showed that the designed sgRNAs successfully induced DNA-double stranded breaks in human SOHLH1 and TEX11 genes that are associated with NOA.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “sequence listing.txt” (˜72 kb), which was created on Jul. 26, 2018, and which is incorporated by reference herein.

SEQ ID NOS: 1-8 are exemplary sgRNAs that can be used for gene therapy in a Soh1h1-KO mouse model.

SEQ ID NOS: 9-12 are exemplary sgRNAs that can be used for gene therapy in a TEX11-KO mouse model.

SEQ ID NOS: 13-16 are exemplary sgRNAs that can be used to target Rosa26 locus for safe harbor gene therapy.

SEQ ID NO: 17 is an exemplary plasmid sequence that one or more sgRNAs can be cloned into via the Bbsl restriction site.

SEQ ID NO: 18 is an exemplary donor template sequence (pUC19 Donor Soh1h1 mCherry PURO-1).

SEQ ID NO: 19 is an exemplary donor template sequence (pUC19 Donor Soh1h1 mCherry PURO-2).

SEQ ID NO: 20 is an exemplary donor template sequence (pUC19 Donor Soh1h1 mCherry TEX11).

SEQ ID NO: 21 is an exemplary donor template sequence (pUC19 Donor Rosa26 PGK-puromycin-T2A-mCherry-T2A-Sohh1h1 cDNA-sv4OpolyA).

SEQ ID NO: 22 is an exemplary donor template sequence (pUC19 Donor Rosa26 PGK-puromycin-T2A-mCherry-T2A-Tex11 cDNA-sv40polyA).

SEQ ID NO: 23 is an exemplary sgRNA sequence that can be used to target SOHLH1.

SEQ ID NO: 24 is an exemplary sgRNA sequence that can be used to target the wild-type locus where the TEX11 c.792+1G->A mutation occurs in humans.

SEQ ID NOS: 25 and 26 are top and bottom strands for the sgRNA of SEQ ID NO: 23.

SEQ ID NOS: 27 and 28 are top and bottom strands for the sgRNA of SEQ ID NO: 24.

### DETAILED DESCRIPTION

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising a nucleic acid molecule” means “including a nucleic acid molecule” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents, and sequences associated with the GenBank® Accession Numbers listed (as of Jul. 26, 2017) are herein incorporated by reference in their entireties.

In order to facilitate review of the various embodiments of the disclosure, the following explanations of specific terms are provided:

### I. Terms

Administration: To provide or give a subject an agent, such as a nucleic acid molecule that corrects a genetic defect, by any effective route. Exemplary routes of administration include, but are not limited to, injection (such as injection into the testis, for example injection into the testicular seminiferous tubules or into the interstitial space, or injection into an ovary).

Cas9: An RNA-guided RNA endonuclease enzyme that can cut DNA. Cas9 has two active cutting sites (HNH and RuvC), one for each strand of the double helix. In some examples, a Cas9 protein includes one or more of the following point mutations: D10A, H840A, N863A.

Cas9 sequences are publicly available. For example, GenBank® Accession Nos. nucleotides 796693..800799 of CP012045.1 and nucleotides 1100046..1104152 of CP014139.1 disclose Cas9 nucleic acids, and GenBank® Accession Nos. NP_269215.1, AMA70685.1 and AKP81606.1 disclose Cas9 proteins. In some examples, the Cas9 is a deactivated form of Cas9 (dCas9), such as one that is nuclease deficient (e.g., those shown in GenBank® Accession Nos. AKA60242.1 and KR011748.1). In certain examples, Cas9 has at least 80% sequence identity, for example at least 85%, 90%, 95%, 98%, or 99% sequence identity to such sequences, and retains the ability to be used in the disclosed methods.

Complementarity: The ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

Contact: Placement in direct physical association, including a solid or a liquid form. Contacting can occur in vitro or ex vivo, for example, by adding a reagent to a sample (such as one containing SSCs, iPSCs, or PGCLCs), or in vivo by administering to a subject.

CRISPRs (clustered regularly interspaced short palindromic repeats): DNA loci containing short repetitions of base sequences. Each repetition is followed by short segments of “spacer DNA” from previous exposures to a virus. CRISPRs are found in approximately 40% of sequenced bacteria genomes and 90% of sequenced archaea. CRISPRs are often associated with cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and cut these exogenous genetic elements in a manner analogous to RNAi in eukaryotic organisms. The CRISPR/Cas system can be used for gene editing (adding, disrupting or changing the sequence of specific genes) and gene regulation. By delivering a Cas9 protein and appropriate guide RNAs into a cell (such as into a somatic cell of the testis, a spermatogonial stem cell (SSC) from the testes, or patient-derived iPSCs), the subject genome can be cut at any desired location.

Downregulated or knocked down: When used in reference to the expression of a molecule, such as a gene or a protein (e.g., a target gene, such as one whose increased expression is associated with NOA or POI), refers to any process which results in a decrease in production of a gene product, but in some examples not complete elimination of the gene product or gene function. In one example, downregulation or knock down does not result in complete elimination of detectable expression or activity. A gene product can be RNA (such as mRNA, rRNA, tRNA, and structural RNA) or protein. Therefore, downregulation or knock down includes processes that decrease transcription of a gene or translation of mRNA and thus decrease the presence of proteins or nucleic acids. The disclosed methods can be used to downregulate any target gene whose expression (such as undesirable increased expression) is associated with NOA or POI.

Downregulation or knock down includes any detectable decrease in the production of a gene product, for example in a somatic cell of the testis, a spermatogonial stem cell (SSC), patient-derived iPSCs, or PGCLCs. In certain examples, using the disclosed methods reduces detectable target protein or nucleic acid expression in a cell by at least 20%, at least 30%, at least 40%, at least 50%, at least 60%, at least 70%, at least 75%, at least 80%, at least 90%, at least 95%, at least 96%, at least 97%, at least 98%, or at least 99% (such as a decrease of 40% to 90%, 40% to 80% or 50% to 95%) as compared to a control (such an amount of protein or nucleic acid expression detected in a corresponding normal cell or sample). In one example, a control is a relative amount of expression in a normal cell (e.g., a non-recombinant somatic cell of the testis, a non-recombinant SSC, or non-recombinant iPSC).

Effective amount: The amount of an agent (such as a recombinant nucleic acid molecule for correcting a genetic defect associated with NOA or POI) that is sufficient to effect beneficial or desired results.

A therapeutically effective amount may vary depending upon one or more of: the subject and disease condition being treated, the weight and age of the subject, the severity of the disease condition, the manner of administration and the like, which can be determined by one of ordinary skill in the art. The beneficial therapeutic effect can include amelioration of a disease, symptom, disorder, or pathological condition; reducing or preventing the onset of a disease, symptom, disorder or condition; and generally counteracting a disease, symptom, disorder or pathological condition. In one embodiment, an “effective amount” is an amount sufficient to increase the sperm recovery rate of a treated male, for example by at least 10%, at least 20%, at least 50%, at least 70%, at least 90%, at least 100%, at least 200%, at least 500% (as compared to no administration of the therapy). In one embodiment, an “effective amount” is an amount sufficient to increase the egg recovery rate of a treated female, for example by at least 10%, at least 20%, at least 50%, at least 70%, at least 90%, at least 100%, at least 200%, at least 500% (as compared to no administration of the therapy).

Expression: The process by which the coded information of a nucleic acid molecule, such as a target nucleic acid molecule is converted into an operational, non-operational, or structural part of a cell, such as the synthesis of a protein (e.g., target protein). Expression of a gene can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

The expression of a nucleic acid molecule or protein can be altered relative to a normal (wild type) nucleic acid molecule or protein (such as in a normal non-recombinant cell). Alterations in gene expression, such as differential expression, include but are not limited to: (1) overexpression (e.g., upregulation); (2) underexpression (e.g., downregulation); or (3) suppression of expression. Alternations in the expression of a nucleic acid molecule can be associated with, and in fact cause, a change in expression of the corresponding protein.

Protein expression can also be altered in some manner to be different from the expression of the protein in a normal (wild type) situation. This includes but is not necessarily limited to: (1) a mutation in the protein such that one or more of the amino acid residues is different; (2) a short deletion or addition of one or a few (such as no more than 10-20) amino acid residues to the sequence of the protein; (3) a longer deletion or addition of amino acid residues (such as at least 20 residues), such that an entire protein domain or sub-domain is removed or added; (4) expression of an increased amount of the protein compared to a control or standard amount (e.g., upregulation); (5) expression of a decreased amount of the protein compared to a control or standard amount (e.g., downregulation); (6) alteration of the subcellular localization or targeting of the protein; (7) alteration of the temporally regulated expression of the protein (such that the protein is expressed when it normally would not be, or alternatively is not expressed when it normally would be); (8) alteration in stability of a protein through increased longevity in the time that the protein remains localized in a cell; and (9) alteration of the localized (such as organ or tissue specific or subcellular localization) expression of the protein (such that the protein is not expressed where it would normally be expressed or is expressed where it normally would not be expressed), each compared to a control or standard.

Controls or standards for comparison to a sample, for the determination of differential expression, include samples believed to be normal (in that they are not altered for the desired characteristic, for example a non-recombinant cell) as well as laboratory values, even though possibly arbitrarily set, keeping in mind that such values can vary from laboratory to laboratory. Laboratory standards and values may be set based on a known or determined population value and can be supplied in the format of a graph or table that permits comparison of measured, experimentally determined values.

Gene Editing: A type of genetic engineering in which a nucleic acid molecule, such as DNA, is inserted, deleted or replaced in the genome of an organism using engineered nucleases, which create site-specific double-strand breaks (DSBs) at desired locations in the genome. The induced double-strand breaks are repaired through nonhomologous end-joining (NHEJ) or homologous recombination (HR), resulting in targeted mutations or repairs. The methods disclosed herein can be used to edit the sequence of one or more target genes associated with NOA or POI. For example, gene editing can be used to correct a genetic mutation in germ cells or somatic cells of the testis (e.g., in one or both one alleles of the mutation), patent-derived iPSCs, or PGCLCs generated from the iPSCs, which result in NOA or other genetic disease.

Gene Silencing: A specific type of gene regulation, namely significantly reducing (e.g., a reduction of at least 90%, at least 95%, or at least 99%) or preventing expression of a gene. Can also be referred to as knocking out gene expression, when the gene is completely silenced. The methods disclosed herein can be used to silence expression of one or more target genes, such as one whose expression is associated with NOA or POI.

Guide sequence: A polynucleotide sequence having sufficient complementarity with a target polynucleotide sequence to hybridize with the target sequence (such as a mutation in TEX11, GCNA, PORCN, MAGEB10, AKAP4, FMR1, SCML2, SOX3, androgen receptor (AR), AFF4, AKAP9, SOHLH1, MCM8, FMR1, or DCAF17) and direct sequence-specific binding of a Cas9 to the target sequence. In some examples, the guide sequence is RNA. In some examples, the guide sequence is DNA. The guide nucleic acid can include modified bases or chemical modifications (e.g., see Latorre et al., Angewandte Chemie 55:3548-50, 2016). In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about, or at least, about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. In some embodiments, a guide sequence is 15-25 nucleotides (such as 18-22 or 18 nucleotides).

The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a target cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

Hemizygous: Having or characterized by one or more genes (as in a genetic deficiency or in an X chromosome paired with a Y chromosome) that have no allelic counterparts. For example, males are normally hemizygous for genes on both sex chromosomes. In some examples, a cell that is hemizygous for a particular mutation is a transformed or recombinant cell (e.g., SSC. iPSC, or PGCLC), such as one that includes a corrected mutation on the X or Y chromosome of a male who only has one X and one Y chromosome. Repair of that mutation on the target chromosome will also be hemizygous. In some examples, a transformed or recombinant cell was homozygous for wild type sequences at a “safe harbor” locus (such as ROSA 26) prior to its transformation with a recombinant nucleic acid molecule. If a transgene is inserted into one allele of the “safe harbor” locus, the transformed or recombinant cells are hemizygous because there is no corresponding sequence on the other allele.

Heterozygous: Refers to an individual, cell, or nucleus, that has two different (e.g., non-identical) alleles for a specific trait, such as a genetic mutation associated with infertility (such as those disclosed herein). In some examples, a cell that is heterozygous for a particular mutation is a transformed or recombinant cell (SSC or iPSC cell). In some examples, such a transformed or recombinant cells was homozygous for the mutation prior to its transformation with a recombinant nucleic acid molecule that corrected the genetic mutation.

Homology-directed repair (HDR): A mechanism to repair double stranded DNA lesions. The methods disclosed herein can be used for HDR of one or more target genes, for example during G2 and S phase of the cell cycle.

Increase or Decrease: A statistically significant positive or negative change, respectively, in quantity from a control value. An increase is a positive change, such as an increase at least 50%, at least 100%, at least 200%, at least 300%, at least 400% or at least 500% as compared to the control value. A decrease is a negative change, such as a decrease of at least 20%, at least 25%, at least 50%, at least 75%, at least 80%, at least 90%, at least 95%, at least 98%, at least 99%, or at least 100% decrease as compared to a control value. In some examples the decrease is less than 100%, such as a decrease of no more than 90%, no more than 95% or no more than 99%.

Induced pluripotent stem cells (iPSCs). iPSCs are derived by reprogramming patient somatic cells (e.g., skin or blood cells) to and pluripotent, embryonic-like state with potential to differentiate into all cell types of the body, including the germ cell lineage that give rise to eggs females and sperm in males.

Isolated: An “isolated” biological component (such as a protein or nucleic acid, or cell) has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Isolated proteins, nucleic acids, or cells (such as somatic cells, SSCs obtained from the testis, iPSCs, PGCLCs, ase well as transformed SSCs, transformed iPSCs or transformed PGCLCs that are heterozygous or hemizygous for the genetic mutation associated with infertility) in some examples are at least 50% pure, such as at least 75%, at least 80%, at least 90%, at least 95%, at least 98%, or at least 100% pure.

Minichromosome maintenance complex component 8 (MCM8): (e.g., OMIM 608187): This gene encodes a protein essential for the initiation of eukaryotic genome replication. In humans, this gene is located on chromosome 20 (20p12.3). Mutations in this gene are associated with premature ovarian failure, POI, and NOA. Exemplary mutations are shown in Table 1.

MCM8 sequences are publically available, for example from the GenBank® sequence database (e.g., Accession Nos. NP_001268449.1, NP_877954.1, NP_001099984.1, and B9FKM7.1 provide exemplary MCM8 protein sequences, while Accession Nos. NM_032485.5, NM_001291054.1, NM_182802.2, and NM_001265899.1 provide exemplary MCM8 nucleic acid sequences). One of ordinary skill in the art can identify additional MCM8 nucleic acid and protein sequences, including MCM8 variants, such as those having at least 80%, at least 85%, at least 90%, at least 92%, at least 95%, at least 98%, or at least 99% sequence identity to these GenBank® sequences. Such MCM8 sequences can be used to generate therapeutic recombinant nucleic acid molecules, such as sgRNAs, to correct a MCM8 mutation.

Modulate: A change in the content of genomic DNA gene. Modulation can include, but is not limited to, gene activation (e.g., upregulation), gene repression (e.g., downregulation), gene deletion, polynucleotide insertion, and/or polynucleotide excision.

Non-homologous end-joining (NHEJ): A mechanism that repairs double stranded breaks in DNA. The methods disclosed herein can be used for NHEJ of one or more target genes.

Non-naturally occurring or engineered: Terms used herein as interchangeably and indicate the involvement of the hand of man. The terms, when referring to nucleic acid molecules or polypeptides indicate that the nucleic acid molecule or the polypeptide is at least substantially free from at least one other component with which they are naturally associated in nature and as found in nature. In addition, the terms can indicate that the nucleic acid molecules or polypeptides is one having a sequence not found in nature.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence (such as a Cas9 coding sequence) if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of recombinant nucleic acid molecule (such as one to correct a genetic defect/mutation associated with NOA or POI).

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide, peptide and protein: Refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

Primordial germ cell-like cells (PGCLCs). PGCLCs are derived from pluripotent stem cells, such as iPSCs, which are in turn derived from patient somatic cells (e.g., skin or blood). PGCLCs from a male subject can give rise to sperm. PGCLCs from a female subject can give rise to eggs.

Promoter: An array of nucleic acid control sequences which direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor). In one example the promoter used is native to the nucleic acid molecule it is expressing (endogenous promoter), for example, is endogenous to the defective NOA associated gene. In one example the promoter used is not native to the nucleic acid molecule it is expressing (exogenous promoter). Exemplary promoters that can be used in with the nucleic acid molecules provided herein include: is a U6, elongation factor 1a (EF1a), CMV, ROSA, Ubiquitin C (UBC), Chicken b-actin (CAAG).

Recombinant or host cell: A cell that has been genetically altered, or is capable of being genetically altered by introduction of an exogenous polynucleotide, such as a recombinant plasmid or vector. Typically, a host cell is a cell in which a recombinant nucleic acid molecule can be propagated and/or its DNA expressed. Such cells can be a somatic cell of the testis or a SSC from the testis. The term also includes any progeny of the subject host cell. It is understood that all progeny may not be identical to the parental cell since there may be mutations that occur during replication. However, such progeny are included when the term “host cell” is used.

Regulatory element: A phrase that includes promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990) which is hereby incorporated by reference in its entirety. Regulatory elements include those that direct constitutive expression of a nucleotide sequence in many types of host cells and those that direct expression of the nucleotide sequence only in certain host cells (e.g., tissue-specific regulatory sequences). A tissue-specific promoter may direct expression primarily in a desired tissue of interest, such as testis. Regulatory elements may also direct expression in a temporal-dependent manner, such as in a cell-cycle dependent or developmental stage-dependent manner, which may or may not also be tissue or cell-type specific.

In some embodiments, a vector provided herein includes one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, CAG promoter, UBC promoter, ROSA promoter, and the EF1α promoter.

Also encompassed by the term “regulatory element” are enhancer elements, such as WPRE; CMV enhancers; the R-U5′ segment in LTR of HTLV-I (Mol. Cell. Biol., Vol. 8(1):466-472, 1988); SV40 enhancer; and the intron sequence between exons 2 and 3 of rabbit β-globin (Proc. Natl. Acad. Sci. USA., 78(3):1527-31, 1981).

Sequence identity/similarity: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Variants of protein and nucleic acid sequences known in the art and disclosed herein are typically characterized by possession of at least about 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Spermatogenesis And Oogenesis Specific Basic Helix-Loop-Helix (SOHLH1): (e.g., OMIM 610224): This gene encodes a testis-specific transcription factor, which is essential for spermatogenesis, oogenesis and folliculogenesis. In humans, this gene is located on chromosome 9 (9q34.3). Mutations in this gene are associated with nonobstructive azoospermia (NOA). An exemplary mutation is c.346-1G>A.

SOHLH1 sequences are publically available, for example from the GenBank® sequence database (e.g., Accession Nos. NP_001095147.1, NP_001012415.2, NP_001001714.1, NP_001178781.1, XP_006918252.1 1, and XP_008968301.1 provide exemplary SOHLH1 protein sequences, while Accession Nos. NM_001101677.1, NM_001012415.2, NM_001001714.1, NM_001191852.1, XM_011724725.1 and XM_008970053.1 provide exemplary SOHLH1 nucleic acid sequences). One of ordinary skill in the art can identify additional SOHLH1 nucleic acid and protein sequences, including SOHLH1 variants, such as those having at least 80%, at least 85%, at least 90%, at least 92%, at least 95%, at least 98%, or at least 99% sequence identity to these GenBank® sequences. Such SOHLH1 sequences can be used to generate therapeutic recombinant nucleic acid molecules, such as sgRNAs, to correct a SOHLH1 mutation.

Spermatogonial stem cell (SSC): SSCs develop from pro-spermatogonia in the testis, and are the early precursor for spermatozoa. SSCs can be divided into more SSCs, or differentiate into spermatocytes, spermatids, and spermatozoa. SSCs can be obtained directly from the testis by testicular biopsy. SSCs or subpopulations of SSCs express the cellular markers CD90, ID4, ITGA6, BMI1, NANOS2, GFRa1, UTF1, CDH1, UCHL1, ZBTB16, SALL4, ENO2, GPR125 and FGFR3, but not CD45. Many of those genes are expressed in mice, monkeys and humans, but some are species specific.

Subject: A mammal, such as a human male or female. In some examples, the subject has a genetic disease that can be treated using gene editing methods provided herein, such as a genetic disease that results in non-obstructive azoospermia (NOA) or premature ovarian insufficiency (POI). Mammals include, but are not limited to, murines, simians, humans, farm animals, sport animals, and pets. In one embodiment, the subject is a non-human mammalian subject, such as a monkey or other non-human primate, mouse, rat, rabbit, pig, goat, sheep, dog, cat, boar, bull, horse, or cow. In some examples, the subject is a laboratory animal/organism, such as a mouse, rabbit, or rat. Tissues, cells and their progeny of a biological entity obtained in vivo or cultured in vitro are also encompassed.

Testis-Expressed Gene 11 (TEX11): (e.g., OMIM 300311): This gene is X-linked (in humans, Xq31.1) and expressed in male germ cells. It is a regulator of crossing-over during meiosis. Mutations in this gene are associated with nonobstructive azoospermia (NOA). Exemplary mutations include c.792+1G->A, c652de1237bp (p218de179aa), c551->G (p.M171V), and c652de1237bp (p.218de179aa).

TEX11 sequences are publically available, for example from the GenBank® sequence database (e.g., Accession Nos. AAK31973.1, AAK31951.1, XP_025228272.1, XP_025131831.1,XP_024844380.1 and NP_001003811.1 provide exemplary TEX11 protein sequences, while Accession Nos. NM_001003811.1, NM_031384.2, NM_031276.2, XM_002700044.4 1, XM_025276046.1 and XM_024284080.1 provide exemplary TEX11 nucleic acid sequences). One of ordinary skill in the art can identify additional TEX11 nucleic acid and protein sequences, including TEX11 variants, such as those having at least 80%, at least 85%, at least 90%, at least 92%, at least 95%, at least 98%, or at least 99% sequence identity to these GenBank® sequences. Such TEX11 sequences can be used to generate therapeutic recombinant nucleic acid molecules, such as sgRNAs, to correct a TEX11 mutation.

Therapeutic agent: Refers to one or more molecules or compounds that confer some beneficial effect upon administration to a subject. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition.

Transduced, Transformed, Transfected: A virus or vector “transduces” a cell when it transfers nucleic acid molecules into a cell. A cell is “transformed” or “transfected” by a nucleic acid transduced into the cell when the nucleic acid becomes stably replicated by the cell, either by incorporation of the nucleic acid into the cellular genome, or by episomal replication.

These terms encompasses all techniques by which a nucleic acid molecule can be introduced into such a cell, including transfection with viral vectors, transformation with plasmid vectors, and introduction of naked DNA by electroporation, lipofection, particle gun acceleration and other methods in the art. In some example the method is a chemical method (e.g., calcium-phosphate transfection or polyethyleneimine (PEI) transfection), physical method (e.g., electroporation, microinjection, particle bombardment), fusion (e.g., liposomes), receptor-mediated endocytosis (e.g., DNA-protein complexes, viral envelope/capsid-DNA complexes) and biological infection by viruses such as recombinant viruses (Wolff, J. A., ed, Gene Therapeutics, Birkhauser, Boston, USA, 1994). Methods for the introduction of nucleic acid molecules into cells are known (e.g., see U.S. Pat. No. 6,110,743). These methods can be used to transduce a cell with the disclosed agents to manipulate its genome.

Transgene: An exogenous gene, for example supplied by a vector (such as a viral vector). In one example, a transgene includes a Cas9 coding sequence (or other therapeutic nucleic acid molecule, such as a gene, coding sequence), for example operably linked to a promoter sequence.

Transgenic: A cell or animal (e.g., human or mouse) carrying a transgene.

Treating, Treatment, and Therapy: Any success or indicia of success in the attenuation or amelioration of a pathology or condition, including any objective or subjective parameter such as abatement or diminishing of symptoms. The treatment may be assessed by objective or subjective parameters; including the results of a physical examination, and other clinical tests, and the like. In one example, treatment using the disclosed methods increases sperm production, or sperm recovery directly from the testes, or both, by at least 10%, at least 20%, at least 25%, at least 50%, at least 75%, at least 90%, at least 100%, at least 200%, at least 300%, at least 500%, or at least 1000%. In one example, treatment using the disclosed methods increases the production of PGCLCs, eggs or sperm from male or female patient derived iPSCs by at least 10%, at least 20%, at least 25%, at least 50%, at least 75%, at least 90%, at least 100%, at least 200%, at least 300%, at least 500%, or at least 1000%.

Upregulated: When used in reference to the expression of a molecule, such as a gene or a protein (e.g., a target gene, such as one whose decreased expression is associated with NOA or POI), refers to any process which results in an increase in production of a gene product. A gene product can be RNA (such as mRNA, rRNA, tRNA, and structural RNA) or protein. Therefore, upregulation includes processes that increase transcription of a gene or translation of mRNA and thus increase the presence of proteins or nucleic acids. The disclosed methods, can be used to upregulate any target of interest, such as one whose downregulation is associated with NOA or POI.

Examples of processes that increase transcription include those that increase transcription initiation rate, those that increase transcription elongation rate, those that increase processivity of transcription and those that decrease transcriptional repression. Gene upregulation can include increasing expression above an existing level. Examples of processes that increase translation include those that increase translational initiation, those that increase translational elongation and those that increase mRNA stability.

Upregulation includes any detectable increase in the production of a gene product. In certain examples, detectable target protein or nucleic acid expression in a cell (such as a somatic cell of the testis, a SSC, or a patient-derived iPSC) increases by at least 20%, at least 30%, at least 40%, at least 50%, at least 60%, at least 70%, at least 75%, at least 80%, at least 90%, at least 95%, at least 100%, at least 200%, at least 400%, or at least 500% as compared to a control (such an amount of protein or nucleic acid expression detected in a corresponding normal cell or sample). In one example, a control is a relative amount of expression in a normal cell (e.g., a non-recombinant somatic cell of the testis or a non-recombinant SSC or a nonrecombinant patient-derived iPSC).

Under conditions sufficient for: A phrase that is used to describe any environment that permits a desired activity. In one example the desired activity is expression of a nucleic acid molecule to correct a genetic defect associated with NOA or POI.

Vector: A nucleic acid molecule into which a foreign nucleic acid molecule can be introduced without disrupting the ability of the vector to replicate and/or integrate in a host cell. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art.

A vector can include nucleic acid sequences that permit it to replicate in a host cell, such as an origin of replication. A vector can also include one or more selectable marker genes (such as antibiotic resistance, or a fluorescent protein), and other genetic elements. An integrating vector is capable of integrating itself into a host nucleic acid. An expression vector is a vector that contains the regulatory sequences to allow transcription and translation of inserted gene or genes.

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as 3rd generation integration-deficient lentiviral vectors) or adeno-associated viral (AAV) vector.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

Certain vectors are capable of directing the expression of genes to which they are operatively-linked. Such vectors are referred to herein as “expression vectors.” Common expression vectors of utility in recombinant DNA techniques are often in the form of plasmids. Recombinant expression vectors can comprise a nucleic acid provided herein (such as a guide RNA to correct a genetic defect (in one or both alleles) associated with NOA, POI, or other genetic disorder, or nucleic acid encoding a Cas9 protein) in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). It will be appreciated by those skilled in the art that the design of the expression vector can depend on such factors as the choice of the host cell to be transformed, the level of expression desired, the size of the transgenic cargo, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

### II. Overview of Several Embodiments

Spermatogenesis, the process that produces sperm in the testes, is amenable to gene therapy because it is a stem cell-based system and occurs in the seminiferous tubules of the testis that can be accessed for infusion of stem cells or other therapeutics (e.g., gene therapy vectors). Oogenesis, the process that produces eggs in the ovary, is not a stem cell-based system. However, skin cells (or other somatic cell types, such as blood) from a male or female mammal can be reprogrammed into patient-derived induced pluripotent stem cells (iPSCs) and differentiated into male or female primordial germ cell-like cells (PGCLCs) that can be transplanted into the testes or ovaries, giving rise to sperm or eggs and live offspring^{5,6}. Pluripotent stem cells can be differentiated, entirely ex vivo, into functional eggs or sperm that gave rise to live offspring^{7,8}. The somatic cell (e.g., skin cell) to iPSC to PGCLC results have been achieved using human cells⁹⁻¹².

Although genetic modification and clonal expansion of SSCs or iPSCs has been shown, its application to infertility-associated genetic variants in patient derived cells has not been demonstrated. The idea that patient-derived SSCs or iPSCs can be genetically modified to repair an infertility-associated mutation and that the repaired cells can give rise to eggs, sperm and offspring with or without passing the genetic modification to progeny is new and inventive. In some examples, the disclosed methods allow gene therapy treatment of an infertile male or female to eliminate infertility and genetically linked comorbid diseases (e.g., diabetes, cancer, neurological deficits) from their entire family lineages, without transmitting the genetic modifications to progeny or future generations.

Gene therapy could be used to repair defects/mutations that cause the NOA phenotype in males, for example by restoring gene function, restoring sperm production, and restoring fertility from the resident germ cells. Gene therapy could be used to repair defects/mutations that cause the POI phenotype in females, for example by restoring gene function, enabling egg production from patient-derived iPSCs. Some single gene mutations that cause NOA directly impact the function of germ cells (e.g., SOHLH1, TEX11, MCM8, FMR1)¹³⁻¹⁷; and some mutations impact the testicular somatic cells that are essential for germ cell maturation (e.g., AR, NR5A1, AFF4, APAP9)¹⁸⁻²¹. Some single gene mutations reduce the size of the pool of follicles in the ovary, leading to POI (e.g., MCM8, DCAF17 (C2orf37), FMR1)²²⁻²⁴. However, there are ethical/societal concerns about doing gene therapy in and around the germline because genetic modifications could be passed to the offspring. Concerns include, but are not limited to: 1) the unborn child does not have the opportunity to consent to the experimental gene therapy and 2) if an unexpected adverse event occurs, it could become a permanent fixture in that family's lineage. This disclosure describes gene therapy methods to treat male infertility that target testicular somatic cells and testicular germ cells and can circumvent the issue of germline transmission. This disclosure provides in vivo Sertoli cell (somatic cell) gene therapy and ex vivo gene therapy followed by transplantation of germline stem cells as examples. Specific applications for 1) autosomal recessive mutations and 2) X-chromosome linked mutations are described.

Gene therapy without germline transmission is non-obvious. Many learned societies (the US National Academy of Sciences; US National Academy of Medicine; British Royal Society and the Chinese Academy of Sciences) have advised against germline gene therapy due to concerns about germline transmission. In fact, the concluding remarks from an international summit on human gene editing organized by those societies (held Dec. 1-3, 2015) included the following statement “3. Clinical Use: Germline. Gene editing might also be used, in principle, to make genetic alterations in gametes or embryos, which will be carried by all of the cells of a resulting child and will be passed on to subsequent generations as part of the human gene pool.”²⁵ It was not obvious to this multidisciplinary collection of world experts that germline gene editing could be accomplished without passing the genetic alterations to the resulting child and subsequent generations. Interestingly, despite these concerns, the National Academies Press published recommendations in 2017 that heritable germline gene editing should be permitted within a robust and effective regulatory framework²⁶. Thus, until the present disclosure, it is not obvious that it is possible to perform germline gene therapy without germline transmission.

The disclose methods allow for treating a man for his infertility, with or without germline transmission, which reduce or eliminate susceptibility to infertility AND other genetically linked diseases in his children and future generations. Male infertility is associated with increased risk of numerous medical comorbidities, including cardiovascular disease, cancer, metabolic syndrome, multiple sclerosis and others²⁷⁻³⁶. As specific examples, mutations in the DCAF17 (C2orf37) gene causes Woodhouse Sakati syndrome, an autosomal recessive multisystem disorder characterized by azoospermia, alopecia, diabetes, deafness, cognitive decline and other features³⁷⁻⁴⁰. Mutations in the MCM8 gene cause nonobstructive azoospermia with maturation arrest (NOA-MA) in mice and men. This is a germ cell defect that is also associated with DNA damage/repair defects and cancer^{22,41-44}. Mutations in the FMR1 gene that is associated with premature ovarian insufficiency (POI) are also associated with mental retardation²⁴.

The disclosed methods for germline gene therapy without germline transmission can reduce societal concerns that germline editing will lead to germline transmission and unforeseen sequelae. The mitigation of this risk may in turn establish safety and feasibility that reduces societal objections and open the door to treatment of a broad spectrum of somatic diseases through the germline by purposeful germline modification. A similar approach can be used through the female germline, but includes genetic modification of patient-derived induced pluripotent stem cells (iPSCs) and differentiating the modified cells into transplantable primordial germ cell-like cells (PGCLCs) or eggs that can be fertilized to produce live offspring^{6,7}. This methodology can also be applied through the male germline^{5,8}.

Provided herein are ex vivo methods for treating a male subject with non-obstructive azoospermia (NOA) or a female subject with premature ovarian insufficiency (POI) caused by one or more genetic mutations. Exemplary genetic defects or mutations include one or more nucleotide and/or amino acid deletions, substitutions, insertions, or combinations thereof. In some examples, the genetic mutation causing the NOA or POI is a homozygous recessive mutation. In some examples, the genetic mutation causing the NOA or POI is a autosomal recessive or sex-chromosome-linked recessive, such as an X-linked recessive mutation. In some examples, the genetic mutation causing the NOA or POI is a dominant mutation. In some examples, the genetic defect causing the NOA or POI includes a mutation in TEX11, GCNA, PORCN, MAGEB10, AKAP4, FMR1, SCML2, SOX3, MCM8, androgen receptor (AR), AFF4, AKAP9, or SOHLH1. In some examples, the subject has a sperm or egg recovery rate of 0% and fertility treatment options are limited. In some examples, the subject has a uniform maturation arrest phenotype (NOA-MA). Exemplary subjects that can be treated include human and veterinary mammals, such as primates, mice, rats, rabbits, bulls, horses, cows, pigs, and sheep.

ex vivo methods for treating a male subject with NOA caused by one or more genetic mutations can include introducing ex vivo (e.g., in culture) a recombinant nucleic acid molecule into spermatogonial stem cells (SSCs) from the testis of the subject, wherein the nucleic acid molecule corrects a genetic mutation causing the NOA (e.g., wherein the nucleic acid molecule corrects one or both alleles of the mutation), and wherein the subject has the genetic mutation. Such methods can include isolating SSCs from the testes, and then culturing the SSCs ex vivo, for example in DMEM alpha, IMDM, or StemPRO. The recombinant nucleic acid molecule is introduced into the SSCs in culture, thereby generating transformed SSCs. In some examples, individual transformed SSCs are obtained (e.g., clones). The transformed SSCs (e.g., transformed SCC clonal population) are screened to identify clones that carry one copy of the corrective transgene (Tg). Selected diploid SSCs with one mutant allele (containing the NOA causing mutation) and one modified allele (carrying the corrective transgene) are expanded in culture, and then transplanted (e.g., introduced, injected) into the testis of the subject. The selected, diploid heterozygous or hemizygous SSCs will regenerate spermatogenesis and produce haploid sperm, 50% of which will carry the mutant allele and 50% of which will carry the therapeutic transgene. In some examples, the same ex vivo approach can be used to transform, screen and select correctly-modified male or female patient iPSCs that can be differentiated into transplantable PGCLCs or eggs or sperm. In some examples, such methods do not transmit the recombinant nucleic acid molecule to progeny of the treated subject. These approaches can be combined with intracytoplasmic sperm injection (ICSI) to produce embryos and preimplantation genetic diagnosis (PGD) to select only embryos that do not carry the therapeutic transgene for transfer into the uterus. The transferred embryos can have a healthy copy of the affected gene from “Mom” so, in the case of recessive diseases, the next generation will be carriers of the mutant allele, but fertile, even in the absence of germline transmission of the transgene.

In some examples, the in vivo or ex vivo method further includes introducing a second recombinant nucleic acid molecule into the somatic cell of the testis, into the SSCs in culture, or into the iPSCs in culture, wherein the second recombinant nucleic acid molecule encodes a Cas9 protein. In such examples, the recombinant nucleic acid molecule used to correct the NOA or POI genetic mutation (e.g., corrects one or both alleles of the mutation) comprises a guide nucleic acid molecule, such as a guide RNA. In some cases, the recombinant nucleic acid molecule is a homologous DNA template used for homology-directed repair.

In some examples, the in vivo or ex vivo method further includes introducing a Cas9 protein into the somatic cell of the testis of the subject, introducing a Cas9 protein into the SSCs in culture, or introducing a Cas9 protein into the iPSCs in culture. In some such examples, the Cas9 protein and the recombinant nucleic acid molecule used to correct the genetic defect are mixed with one another, prior to introducing into the somatic cell of the testis of the subject, into the SSCs in culture, or into the iPSCs in culture.

The recombinant nucleic acid molecule used to correct the NOA or POI genetic defect (or the nucleic acid molecule encoding a Cas9 protein) can include other elements, such as one or more selectable markers (e.g., puromycin or other antibiotic resistance) or reporter molecules (e.g., fluorescent protein). In some examples, the recombinant nucleic acid molecule used to correct the NOA or POI genetic defect (or the nucleic acid molecule encoding a Cas9 protein) is operably linked to a promoter, such as a UBC, ROSA, EF1a, chicken β actin, PGK or U6 promoter. In some examples, the promoter is a cell-type-specific promoter, such as VASA, DAZL or ZBTB16 for germ cells and SOX9 for Sertoli cells. In some examples, the endogenous promoter of the defective gene is used, such that a promoterless nucleic acid molecule used to correct the NOA, POI, or other genetic disorder will be precisely placed, using CRISPR/Cas9, adjacent to the endogenous promoter. In some examples, the recombinant nucleic acid molecule used to correct the NOA or POI genetic defect further encodes for a Cas9 protein (that is, a single nucleic acid molecule includes both). In some examples, the recombinant nucleic acid molecule comprises a guide nucleic acid molecule, such as a guide RNA including a sequence that targets the genetic defect.

In some examples, the recombinant nucleic acid molecule used to correct the NOA, POI, or other genetic disorder targets an endogenous native locus associated with NOA, POI, or other genetic disorder, respectively. In other examples, the recombinant nucleic acid molecule used to correct the NOA, POI, or other genetic disorder mutation targets a safe harbor locus, such as a ROSA26, adeno-associated virus site 1 (AAVS1), chemokine (CC motif) receptor 5 (CCRS), or hH11 locus.

The recombinant nucleic acid molecule used to correct the NOA, POI, or other genetic disorder mutation, or the nucleic acid molecule encoding a Cas9 protein can be part of a vector, such as a viral vector or plasmid vector. Exemplary viral vectors that can be used include an adenovirus, adeno-associated virus, or lentivirus. Thus, in some examples, introducing the nucleic acid molecules into the somatic cell of the testis, into the SSCs in culture, or into patient-derived iPSCs in culture, includes the use of viral vectors. In some examples, introducing the nucleic acid molecules into the somatic cells of the testis, into the SSCs in culture, or into patient-derived iPSCs in culture, utilizes naked nucleic acid molecules (such as naked DNA), for example by using polyethyleneimine (PEI) or electroporation to facilitate entry of the nucleic acid molecules into the target cell.

### III. Methods for Treating NOA and POI

Methods are provided for treating non-obstructive azoospermia (NOA) in a male subject (e.g., infertile subject), wherein the NOA is caused by a genetic mutation, such as one that causes a germ cell development defect and has a recessive or dominant mode of inheritance. Thus, such mutations may affect development of sperm or sperm precursor cells, such as primordial germ cells, pre-spermatogonia, pro-spermatogonial, gonocytes, spermatogonial stem cells, undifferentiated spermatogonia, differentiated spermatogonia, spermatocytes, and/or spermatids). In some examples, the genetic mutation that causes NOA also causes another comorbid disease, such as cancer (such as a cancer of the breast, lung, liver, or colon), diabetes, cardiovascular disease, metabolic syndrome, multiple sclerosis, deafness, or a neurological deficit, such as Woodhouse Sakati syndrome and mental retardation. Thus, in some examples, the methods treat not only the infertility, but the comorbid disease as well. In one example, such methods can include introducing one or more recombinant nucleic acid molecules into spermatogonial stem cells (SSCs) from the testes of the subject, resulting transformed SSCs, wherein the nucleic acid molecule corrects the genetic mutation causing the NOA (e.g., wherein the nucleic acid molecule corrects one allele of the mutation, or both alleles of the mutation). Transformed SSCs that are heterozygous or hemizygous for the genetic mutation can be isolated or purified, thereby generating isolated transformed SSCs. The isolated transformed SSCs that are heterozygous or hemizygous for the genetic mutation are introduced or transplanted the into the male subject, thereby treating NOA (and in some examples also the comorbid disease) in the subject. In another example, the method includes introducing one or more recombinant nucleic acid molecules into induced pluripotent stem cells (iPSCs) of the male subject, wherein the nucleic acid molecule corrects the genetic mutation causing the NOA (e.g., wherein the nucleic acid molecule corrects one allele of the mutation, or both alleles of the mutation), thereby generating transformed iPSCs. Transformed iPSCs that are heterozygous or hemizygous for the genetic mutation can be isolated or purified, thereby generating isolated transformed iPSCs. The isolated transformed iPSCs that are heterozygous or hemizygous for the genetic mutation are differentiated into primordial germ cell-like cells (PGCLCs), which are then either (1) transplanted or introduced into (e.g., via injection) the testes of the male subject (e.g., into the seminiferous tubules, which regenerates spermatogenesis in vivo, e.g., produces sperm in the testes), or (2) differentiated into sperm in vitro, thereby treating NOA (and in some examples also the comorbid disease) in the subject.

In another example, NOA is treated in a male subject by introducing a recombinant nucleic acid molecule into a somatic cell of the testis of the male subject (ex vivo or in vivo), wherein the NOA is caused by a genetic mutation, such as one that causes a germ cell development defect and has a recessive or dominant mode of inheritance. Thus, such mutations may affect development of sperm or sperm precursor cells, such as primordial germ cells, pre-spermatogonia, pro-spermatogonial, gonocytes, spermatogonial stem cells, undifferentiated spermatogonia, differentiated spermatogonia, spermatocytes, and/or spermatids). In some examples, the genetic mutation that causes NOA also causes another comorbid disease, such as cancer (such as a cancer of the breast, lung, liver, or colon), diabetes, cardiovascular disease, metabolic syndrome, multiple sclerosis, deafness, or a neurological deficit, such as Woodhouse Sakati syndrome and mental retardation. Thus, in some examples, the methods treat not only the infertility, but the comorbid disease as well. In some examples, the nucleic acid molecule corrects a genetic mutation causing the NOA, but the recombinant nucleic acid molecule is not transmitted to progeny of the treated male subject. In some examples, such a method is performed in vivo, and introducing the recombinant nucleic acid molecule into the somatic cell of the testis includes injecting the recombinant nucleic acid molecule into the testicular seminiferous tubules or into the interstitial space. Exemplary somatic cells include Sertoli cells, peritubular myoid cells, Leydig cells, and combinations thereof.

Methods for treating premature ovarian insufficiency (POI) (also known as premature ovarian failure (POF), primary ovarian insufficiency, and primary ovarian failure) in a female subject (e.g., infertile subject), wherein the POI is caused by a genetic mutation, such as one that causes a germ cell development defect and has a recessive or dominant mode of inheritance. Thus, such mutations may affect development of eggs or egg precursor cells, such as primordial germ cells, oogonia or developing oogonia (eggs) in developing follicles including primordial follicles, secondary follicles, tertiary follicles, antral follicles or Graffian follicles. In some examples, the genetic mutation that causes NOA also causes another comorbid disease, such as cancer (such as a cancer of the breast, lung, liver, or colon), diabetes, cardiovascular disease, metabolic syndrome, multiple sclerosis, deafness, or a neurological deficit, such as Woodhouse Sakati syndrome and mental retardation. Thus, in some examples, the methods treat not only the infertility, but the comorbid disease as well. In one example, the method includes introducing one or more recombinant nucleic acid molecules into induced pluripotent stem cells (iPSCs) of the female subject, wherein the nucleic acid molecule corrects the genetic mutation causing the POI (e.g., wherein the nucleic acid molecule corrects one allele of the mutation, or both alleles of the mutation), thereby generating transformed iPSCs. Transformed iPSCs that are heterozygous or hemizygous for the genetic mutation can be isolated or purified, thereby generating isolated transformed iPSCs. The isolated transformed iPSCs that are heterozygous or hemizygous for the genetic mutation are differentiated into primordial germ cell-like cells (PGCLCs), which can be mixed with fetal gonadal cells and then either (1) transplanted or introduced into the ovary of the female subject (this regenerates oogenesis in vivo, e.g., produces eggs in the ovary), or (2) differentiated into eggs in vitro, thereby treating POI in the subject. The resulting in vivo-derived eggs can be collected from the ovaries of the treated female subject; or the in vitro-derived eggs from the treated female subject are fertilized with sperm to produce embryos.

In some examples, the male subject to be treated has a sperm recovery rate of 50% or less, such as less than 40%, less than 30%, less than 20%, less than 10%, less than 5%, less than 1%, or even 0% (for example as compared to an amount of sperm obtained using testicular sperm extraction (TESE) from a normal subject). In some examples, the male subject to be treated has azoospermia (no sperm in the ejaculate). In some examples, the male subject to be treated has oligospermia (<15 million sperm/ml ejaculate). In some examples, the male subject to be treated has a uniform maturation arrest phenotype (NOA-MA). In some examples, the female subject to be treated has an egg recovery rate of 50% or less, such as less than 40%, less than 30%, less than 20%, less than 10%, less than 5%, less than 1% or even 0% (for example as obtained using oocyte retrieval). Exemplary subjects that can be treated include human and veterinary mammals, such as primates, mice, rats, rabbits, bulls, horses, cows, pigs, and sheep.

The disclosed methods can include additional steps. In some examples, cells to be transformed with the therapeutic recombinant nucleic acid molecule are obtained from the subject to be treated, prior to introducing the recombinant nucleic acid molecule. For example, prior to introducing the recombinant nucleic acid molecule into the isolated SSCs, the method can include obtaining the SSCs from the testis of the male subject. In some examples, the method also includes culturing ex vivo (for example in the presence of a growth media and nutrients) the isolated SSCs obtained from the testis prior to introducing the recombinant nucleic acid molecule. Alternatively, prior to introducing the recombinant nucleic acid molecule into the iPSCs, the method can include obtaining somatic cells (e.g., skin cells or blood cells) (e.g., from the male or female subject to be treated); and contacting the mammalian somatic cells with appropriate reagents (such as transformation with nucleic acid molecules encoding Oct4, Klf4, Sox2, and Glisl) to reprogram them into patient-derived iPSCs of the male or female subject, wherein the iPSCs are then transformed with the therapeutic recombinant nucleic acid molecule to correct the genetic mutation. To reprogram the somatic cells into iPSCs, a commercial kit can be used (e.g., Simplicon™ RNA reprogramming Kit, SCR549, 550, Millipore). Briefly, patient-derived somatic cells (e.g., skin cells or blood cells, such as fibroblasts) are grown in appropriate media and serum (e.g., DMEM+10% FBS+1× glutaMax), for example for at least 12 hours, at least 1 day, such as at least 2 days. The cultured somatic cells can be pretreated with B 18R protein and transfected with VEE-OSK-iG and B18R RNAs (Part no. CS210583 and CS210584), which contains Oct4, Klf4, Sox2, Glisl, and puromycin-resistant genes. Subsequently (e.g., at about day 3), puromycin is added to the culture (concentration can be adjusted depending on cell survival). The media is changed to MEF-CM (AR 005, R&D systems)+FGF-2 (10 ng/mL) at about day 11, and iPSC colonies picked and expanded at about day 25. In some examples, the method also includes culturing ex vivo (for example in the presence of a growth media and nutrients) the iPSCs of the male or female subject prior to introducing the recombinant nucleic acid molecule.

The methods can also include culturing or growing or expanding (e.g., clonally expanding) the transformed cells ex vivo (for example in the presence of a growth media and nutrients). For example, the methods can include culturing ex vivo the isolated transformed SSCs prior to introducing the transformed SSCs that are heterozygous or hemizygous for the genetic mutation into the male subject.

In some examples, the method includes culturing ex vivo the isolated transformed iPSCs prior to differentiating them into PGCLCs. In some examples, the methods include screening the transformed cells, to identify cells that have been transformed. For example, the recombinant nucleic acid molecule can encode a detectable protein (e.g., fluorescent protein, such as eGFP, or GFP), which allows for identification and selection of the transformed cells, for example by flow cytometry, and/or can encode a selectable marker (such as antibiotic resistance, such as puromycin resistance), which allows for identification and selection of the transformed cells, for example by growth in the antibiotic. In some examples, the growth media includes an antibiotic or other reagent that allows for selection of transformed cells.

The step of isolating the transformed SSCs or the transformed iPSCs that are heterozygous or hemizygous for the genetic mutation can include selecting individual transformed SSCs or individual transformed iPSCs (such as an individual clone or individual cell), and then genotyping the individual transformed SSCs or the individual transformed iPSCs (for example using nucleic acid sequencing). Individual transformed SSCs or individual transformed iPSCs that are heterozygous or hemizygous for the genetic mutation are identified and selected.

The step of differentiating the isolated transformed iPSCs into PGCLCs can be performed using the methods of Hayashi et al. (Cell, 2011. 146(4):519-32), Irie et al. (Cell, 2015. 160(1-2):253-68), and Sasaki et al. (Cell Stem Cell, 2015. 17(2):178-94). In one example, mouse iPSCs are maintained on mouse embryonic feeders (MEFs) in N2B27 medium with 2i+LIF (MAPK inhibitor (PD0325901, 0.4 mM), a GSK3 inhibitor (CHIR99021, 3 mM) and leukemia inhibitory factor (LIF, 1000 u/mL)). These cells are then induced into Epiblast-like cells (EpiLCs) by culturing in N2B27 medium with Activin A (20 ng/mL), bFGF (12 ng/mL), and KSR (1%) for 2 days. EpiLCs are then induced into mPGCLCs under low-binding condition (using low-cell-binding U-bottom 96-well lipidure-coat plate) by passaging EpiLCs into a GK15 medium (GMEM with 15% KSR, 0.1 mM Non-essential amino acid (NEAA), 1 mM sodium pyruvate, 0.1 mM 2-mercaptoethanol, 100 U/mL penicillin, 0.1 mg/mL streptomycin, and 2 mM L-glutamine) in the presence of BMP4 (500 ng/mL), LIF (1000 u/mL), SCF (100 ng/mL), BMP8b (500 ng/mL), and EGF (50 ng/mL) and incubate for 4-6 days. Mouse PGCLCs are then enriched by fluorescence-activated cell sorting (FACS) for Integrin-b3 and SSEA1-positive cells for downstream spermatogenesis or oogenesis. In another example, human IPSCs are maintained on irradiated MEF (mouse embryonic fibroblast) feeder under 4i condition (inhibitors for MAPK, GSK3, p38 and JNK). Prior to hPGCLC induction, the culture is pre-induced by passaging onto vitronectin/gelatin-coated plates in N2B27 medium with 1% KSR, bFGF (10 ng/mL), TGF-b1 (1 ng/mL), or activin A (20 ng/mL) and ROCK inhibitor (10 mM) and incubated for 2 days. The PGCLC induction is performed the same way as in mice (by culturing in BMP2/4, LIF, SCF, EGF and ROCK-i for 4-6 days). Briefly, day 2 after preinduction, cells are cultured in the low-binding condition in GK15 medium with BMP4 or BMP2 (500 ng/mL), human LIF (1 mg/mL), SCF (100 ng/mL), EGF (50 ng/mL), and ROCK inhibitor (10 mM). In yet another example, human iPSCs are induced into iMeLCs (induced-mesenchymal cell-like cells) by plating hiPSCs onto a human plasma fibronectin (Millipore, FC010)-coated 12-well plate in GK15 medium. To induce iMeLCs, activinA, CHIR and ROCK-I are added to the medium to a final concentration of 50 ng/mL, 3 μM and 10 μM, respectively, and incubated for 2 days. The iMeLCs are then induced into hPGCLCs the same way as described above for mice. Briefly, the iMeLCs were cultured in a low-binding condition in GK15 medium with LIF (1,000 U/mL), BMP4 (200 ng/mL), SCF (100 ng/mL), EGF (50 ng/mL) and ROCK inhibitor (10 μM) for 4-6 days.

The step of generating sperm or eggs from the PGCLCs can be performed using the methods of Hayashi et al. (Cell, 2011. 146(4):519-32), Hayashi, et al. (Science, 2012. 338(6109): p. 971-5), Zhao et al. (Stem Cell Reports, 2018. 10(2):509-523), Zhou et al. (Cell Stem Cell, 2016. 18(3):330-40), and Hikabe et al. (Nature, 2016. 539(7628):299-303). In one example, PGCLCs generated from the iPSCs are transplanted into a mammal, such as a human or mouse, for in vivo spermatogenesis. For example, male PGCLCs can be FACS sorted for Integrin-b6 and SSEA-1-positive cells to enrich for PGCLCs. The PGCLC can be transplanted into the testes ovia efferent ductules to generate sperm, for example at 10,000 cells/testis. Sperm can be recovered after transplantation. In one example, PGCLCs generated from the iPSCs are cultured ex vivo for in vitro spermatogenesis. Male PGCLCs can be cultured in 1:1 ratio with dissociated testicular cells in culture media, such as aMEM supplemented with 10% KSR with retinoic acid (e.g., about 10⁻⁶ M), BMP-2/4/7 (e.g., about 20 ng/mL each), and activin A (e.g., about 100 ng/mL) for meiosis initiation (e.g., about 6 days). For meiosis completion, the culture is supplemented with testosterone (e.g., about 10 μM), FSH (e.g., about 200 ng/mL), and BPE (e.g., about 50 μg/mL), for example for 8 days. Round-spermatid-like cells are the final products and can be used to fertilize eggs and to generate offspring.

In one example, spermatogonium-like cells (SLCs) can be generated from iPSCs, and the product of SLCs (e.g., round spermatids) can be used to fertilize eggs. Briefly, human iPSCs can be differentiated into PLZF-positive spermatogonium-like cells (SLCs) by culturing in aMEM with 2 mM L-glutamine, 13 Insulin-Transferrin-Selenium-X, 0.2% BSA orsubstituted by 0.2%-3% KSR XenoFree CTS, 1 ng/mL recombinant human basic fibroblast growth factor(bFGF), 20 ng/mL recombinant human GDNF, 0.2% chemically defined lipid concentrate, and 200 mg/mL vitamin C, for example for 12 days.

In one example, PGCLCs generated from the iPSCs are transplanted into an ovary, such as a human ovary. In one example, PGCLCs generated from the iPSCs are used to generate a reconstituted ovary, female PGCLCs can be co-cultured in 1:10 ratio with female gonadal somatic cells under low-binding condition (e.g., in GK15 medium for 2 days) before transplanting into the ovary (e.g., under the ovarian bursa of mice). The transplanted ovary is recovered at 4 weeks after transplantation to retrieve oocytes for subsequent in vitro maturation. in vitro oogenesis can be performed as follows. To generate oocytes in vitro, reconstituted ovaries can be transferred onto Transwell-COL membranes soaked in aMEM-based IVDi medium (aMEM with 2% FCS, 150 μM ascorbic acid, 1× Glutamax, 1× penicillin/streptomycin and 55 μM 2-mercaptoethanol) for 4 days. Then the medium can be changed into StemPro-34-based IVDi medium (StemPro-34 SFM with 10% FCS, 150 μM ascorbic acid, 1× Glutamax, 1× penicillin/streptomycin and 55 μM 2-mercaptoethanol) with ICI182780 (500nM) added to the medium at day 7-10 of culture. At 21 days, individual secondary-follicle-like structures (2FLs) can be manually dissociated from the culture. To stimulate granulosa cell growth, the single 2FLs can be placed on the Transwell-COL membranes soaked in IVG-aMEM medium (aMEM supplemented with 5% FCS, 2% polyvinylpyrrolidone, 150 μM ascorbic acid, 1× Glutamax, 1× penicillin/streptomycin, 100 μM 2-mercaptoethanol, 55 μg/mL sodium pyruvate, 0.1 IU/mL follicule-stimulating hormone, 15 ng/mL BMP15 and 15 ng/mL GDF9). At 2 days of culture, BMP15 and GDF9 can be withdrawn from the medium. At 11 days of culture, cumulus—oocyte complexes grown on the membrane can be retrieved for in vitro maturation.

The methods can further include generating embryos from sperm obtained from the treated males (either sperm generated in vivo or ex vivo), or eggs from the treated females (either eggs generated in vivo or ex vivo). In some examples, sperm from the treated male is obtained from ejaculate, the testis, or the excurrent duct system of the testis (e.g., efferent ducts, epididymis, vas deferens). In one example, sperm from the treated male subject is introduced into a female egg, for example ex vivo (e.g., via IVF), thereby fertilizing the egg and generating one or more embryos. In some examples, the egg does not have the genetic defect present in the male from whom sperm was obtained. That is, the egg is wild-type (+/+) at that allele. The resulting embryos are analyzed for the presence of the recombinant nucleic acid molecule, for example using preimplantation genetic diagnosis (PGD). In some examples, for example if the genetic mutation that causes NOA is a recessive mutation, embryos that do not include the recombinant nucleic acid molecule (i.e., are not transgenic, but are heterozygous for the mutation, +/−, one mutant allele (−) is from the sperm, and one wild type functional allele (+) from the egg) are selected, and can be implanted into a uterus to establish a pregnancy. Thus, in such examples, the recombinant nucleic acid molecule is not transmitted to progeny of the treated male subject. Offspring resulting from such embryos are heterozygous for the genetic defect present in the male with NOA, and are thus carriers of the genetic infertility-associated allele from Dad, but the offspring are fertile as they are heterozygous for the mutation (instead of homozygous). The genetic infertility-associated allele will be diluted in each subsequent generation and eventually eliminated from the family lineage (assuming non-consanguineous partners). In other examples, for example if the genetic mutation that causes NOA is a dominant mutation, embryos that include the recombinant nucleic acid molecule (i.e., are transgenic on one allele of the affected locus (Tg, inherited from the gene therapy-treated Dad)), and are WT on the other allele of the affected locus (+, inherited from Mom), are selected, and can be implanted into a uterus to establish a pregnancy. In such examples, the recombinant nucleic acid molecule is transmitted to progeny of the treated male subject. Offspring resulting from such embryos are Tg or gene edited at the allele that had the dominant genetic mutation present in the male with NOA, and are will not have the genetic disease nor will they be carriers of the genetic infertility-associated allele from Dad. Thus, the pathogenic infertility-associated mutation is eliminated from the family lineage in one generation.

In another example, one or more eggs from the treated female subject are fertilized with sperm, for example ex vivo (e.g., via IVF), thereby fertilizing the egg(s) and generating one or more embryos. Thus, in some examples, eggs are obtained or harvested from the treated female. In some examples, the sperm used to fertilize the egg does not have the genetic defect present in the female. That is, the sperm is wild-type (+) at that allele. The resulting embryos are analyzed for the presence of the recombinant nucleic acid molecule, for example using PGD. In some examples, such as if the genetic mutation that causes POI is a recessive mutation, embryos that do not include the recombinant nucleic acid molecule (i.e., are not transgenic, but are heterozygous for the mutation, −/+, as one mutant allele (−) is from the egg, and the other wild type functional allele (+) from the sperm) are selected, and can be implanted into a uterus to establish a pregnancy. Thus, in such examples, the recombinant nucleic acid molecule is not transmitted to progeny of the treated female subject. Offspring resulting from such embryos are heterozygous for the genetic defect present in the female with POI, and are thus carriers of the genetic infertility-associated allele from Mom, but the offspring are fertile as they are heterozygous for the mutation (instead of homozygous). The genetic infertility-associated allele will be diluted in each subsequent generation and eventually eliminated from the family lineage (assuming non-consanguineous partners). In other examples, for example if the genetic mutation that causes POI is a dominant mutation, embryos that include the recombinant nucleic acid molecule (i.e., are transgenic, and are WT at the affected locus, Tg/+, as the mutant allele is corrected in the egg (Tg), and the other wild type functional allele (+) is from the sperm) are selected, and can be implanted into a uterus to establish a pregnancy. Thus, in such examples, the recombinant nucleic acid molecule is transmitted to progeny of the treated female subject. Offspring resulting from such embryos are Tg at the allele that had the dominant genetic mutation present in the female with POI, and will not have the genetic disease nor will they be carriers of the genetic infertility-associated allele from Mom. The US consolidated appropriations act of 2016 includes language that specifically prohibits the FDA from receiving applications that would result in the production of a genetically modified human embryos. However, in February 2017, the National Academy of Sciences Committee on Human Gene Editing advised that although germline genome editing trials must be approached with caution, caution does not mean prohibition (www8.nationalacademies.org/onpinews/newsitem.aspx?RecordID=24623). Thus, future government policies may be more accepting of purposeful germline modifications that result in passage of genetic changes to progeny

The recombinant nucleic acid molecule(s) are introduced into the appropriate cells in an effective amount, for example using infection/transfection/transduction methods (e.g., using polyethyleneimine (PEI)). In some examples, the recombinant nucleic acid molecule can correct at least one allele of a mutated gene (e.g., if the associated disease has a recessive mode of inheritance) in the resulting transformed cell. In some examples, the recombinant nucleic acid molecule, such as a cDNA encoding a therapeutic gene, can express a protein that is missing or downregulated in the resulting transformed cell. In some examples, the recombinant nucleic acid molecule includes a recombinant DNA template to direct homology directed modification of the treated subject's genome. The recombinant nucleic acid molecule can be part of a single vector, or divided into multiple vectors. Exemplary vectors include plasmid vectors, and viral vectors (e.g., adenovirus, adeno-associated virus, or lentivirus).

The recombinant nucleic acid molecule (and in some example also a Cas9 protein) targets an endogenous native locus associated with NOA or POI (or other disorder), or targets or a safe harbor locus (such as Rosa26, adeno-associated virus site 1 (AAVS1), chemokine (CC motif) receptor 5 (CCRS), or hH11).

In some examples, the recombinant nucleic acid molecule(s) further includes a selectable marker or reporter gene, such as antibiotic resistance (e.g., puromycin, neomycin, ampicillin, kanamycin), a fluorescent protein (e.g., luciferase, GFP, eGFP), or both. The recombinant nucleic acid molecule(s) can be operably linked to a promoter (such as U6, elongation factor 1a (EF1a), CMV, ROSA, UBC, or chicken b-actin). The recombinant nucleic acid molecule can also include Cas9 coding sequence, such as a DNA or RNA Cas9 sequence. Such a Cas9 coding sequence can be part of the recombinant nucleic acid molecule(s) that corrects the genetic defect (e.g., part of a single vector), or can be a separate molecule (e.g., the nucleic acid molecules can be on separate vectors). The recombinant nucleic acid molecule that corrects the genetic defect can include a guide nucleic acid molecule (e.g., guide ribonucleic acid (RNA) molecule or guide RNA coding sequence).

In some examples, the methods further include introducing a Cas9 protein or Cas9 encoding nucleic acid molecule into the SSCs from the testis of the male subject, introducing a Cas9 protein or Cas9 encoding nucleic acid molecule into the iPSCs of the male or female subject, or introducing a Cas9 protein or Cas9 encoding nucleic acid molecule into the somatic cell of testis of the male subject. In some examples, the Cas9 protein and the recombinant nucleic acid molecule are complexed to one another, prior to introducing into SSCs from the testis of the male subject, the iPSCs of the male or female subject, or the somatic cell of testis of the male subject.

The genetic mutation that causes NOA or POI can be a gene on the X or Y chromosome, such as a mutation in a TEX11, GCNA, PORCN, MAGEB10, AKAP4, FMR1, SCML2, or SOX3 gene. In one example, the genetic mutation that causes NOA is a mutation in the androgen receptor (AR), AFF4 or AKAP9 gene. In one example, the genetic mutation that causes POI is a mutation in the MCM8, FMR1, or DCAF17 gene. In some examples, the genetic mutation that causing infertility has a dominant mode of inheritance, which can be corrected using the disclosed methods by genetic modification of the germline with germline transmission to progeny. In some examples, the genetic mutation that causing infertility has a recessive mode of inheritance, which can be corrected using the disclosed methods by genetic modification of the germline without germline transmission to progeny.

Thus, provided are methods for treating infertility-associated mutations; homozygous recessive or sex-linked recessive mutations; which can be used to achieve germline gene editing with or without germline transmission. It was not previously known how to achieve germline gene therapy for infertility without transmission of the therapeutic genetic sequences to progeny. In addition, it was not previously known how achieve to germline gene therapy without germline transmission to treat the infertility of individual men or women and also reduce or eliminate infertility and genetically linked comorbid disease susceptibility from their children and subsequent generations. Thus, pathogenic alleles can be essentially eliminated from the entire family lineage by treating the infertility of a single individual. Purposeful germline modification can also be used with the disclosed methods to treat or eliminate the other genetic diseases that are associated with infertility, such as FMR1 (male and female infertility; mental retardation), DCAF17 (male and female infertility; Woodhouse-Sakati syndrome characterized by diabetes, alopecia, neurological problems and other phenotypes).

### IV. Expression of Recombinant Nucleic Acid Molecules

The disclosed methods can be used to correct a genetic defect associated with NOA and POI, for example using CRISPR/Cas9 gene editing techniques and/or transgenic techniques. Such methods can be performed ex vivo (such as in cell culture), or in vivo (such as in a male or female mammal). In some examples, such methods modulate (e.g., increase or decrease) expression of one or more target genes, such as AR. For example, by using a transgene or by correcting a genetic defect, a particular target gene may be up- or down-regulated.

Nucleic acid sequences used to correct a genetic defect associated with NOA or POI (or a nucleic acid molecule encoding a Cas9 protein) can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al.,Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. While chemical synthesis of DNA may be limited to sequences of about 100 bases, longer sequences may be obtained by ligating shorter sequences.

In one example, a recombinant nucleic acid molecule used to correct a genetic defect associated with NOA, POI, or other genetic disorder (or a nucleic acid molecule encoding a Cas9 protein) is inserted into a vector, such as a plasmid, virus or other vehicle that can be manipulated to allow insertion or incorporation of sequences into cells and, in some cases, into the cell's genome and can be expressed in somatic cells of the testis, SSCs from the testis, or male of female patient-derived iPSCs. The vector can encode a selectable marker, such as a fluorescent reporter gene, a thymidine kinase gene or puromycin resistance gene, and the like, for example to allow for detection or selection of genetically modified (e.g., transformed) somatic cells or SSCs of the testis or male- or female-patient-derived iPSCs, for example by drug selection or by using FACS.

Nucleic acid molecules used to correct a genetic defect associated with NOA, POI, or other genetic disorder (or a nucleic acid molecule encoding a Cas9 protein) can be operatively linked to expression control sequences. Exemplary expression control sequences include, but are not limited to appropriate promoters, enhancers, transcription terminators, and if appropriate, a start codon (i.e., ATG) in front of the nucleic acid molecule used to correct a genetic defect associated with NOA, POI, or other genetic disorder (or a nucleic acid molecule encoding a Cas9 protein).

Viral vectors can also be prepared that include a recombinant nucleic acid molecule used to correct a genetic defect associated with NOA, POI, or other genetic disorder (or a nucleic acid molecule encoding a Cas9 protein). Exemplary viral vectors include polyoma, SV40, adenovirus, vaccinia virus, adeno-associated virus, lentivirus, herpes viruses including HSV and EBV, Sindbis viruses, alphaviruses and retroviruses of avian, murine, and human origin. Baculovirus (Autographa californica multinuclear polyhedrosis virus; AcMNPV) vectors can be used. Other suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors and poliovirus vectors. Specific exemplary vectors are poxvirus vectors such as vaccinia virus, fowlpox virus and a highly attenuated vaccinia virus (MVA), adenovirus, baculovirus and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Other viral vectors that can be used include other DNA viruses such as herpes virus and adenoviruses, and RNA viruses such as retroviruses and polio.

Viral vectors that include a recombinant nucleic acid molecule used to correct a genetic defect associated with NOA, POI, or other genetic disorder (or a nucleic acid molecule encoding a Cas9 protein) can include at least one expression control element operationally linked to the nucleic acid sequence. The expression control elements are inserted in the vector to control and regulate the expression of the nucleic acid molecule. Examples of expression control elements of use in these vectors includes, but is not limited to, lac system, operator and promoter regions of phage lambda, yeast promoters and promoters derived from polyoma, adenovirus, retrovirus or SV40. In one example the promoter is CMV or U6. Some exemplary promoters that can be used include CMV, SP6, U6, ROSA, elongation factor 1a (EF1a), Chicken β-actin, phosphoglycerate kinase (PGK) and ubiquitin C (UBC). Additional operational elements include, but are not limited to, leader sequence, termination codons, polyadenylation signals and any other sequences necessary for the appropriate transcription and subsequent translation of the recombinant nucleic acid molecule used to correct a genetic defect associated with NOA, POI, or other genetic disorder in the target cells. The expression vector can contain additional elements necessary for the transfer and subsequent replication of the expression vector containing the nucleic acid sequence in the cells. Examples of such elements include, but are not limited to, origins of replication and selectable markers.

Methods of introducing the recombinant nucleic acid molecule used to correct a genetic defect associated with NOA, POI, or other genetic disorder (or a nucleic acid molecule encoding a Cas9 protein) into a somatic cell of the testis (such as Sertoli cell, peritubular myoid cell, Leydig cell, or combinations thereof) or SSCs from the testis or patient-derived iPSCs, can include using calcium phosphate coprecipitates, PEI, mechanical procedures such as microinjection, electroporation, insertion of a plasmid encased in liposomes, or viral vectors.

In one example, a CRISPR/Cas9 system is used to correct a genetic defect or mutation associated with NOA, POI, or other genetic disorder. CRISPR/Cas9 generally includes three components: (1) a Cas9 protein or RNA whose expression can be driven by a promoter, such as EF1a or UBC, (2) a guide nucleic acid molecule, such as RNA (sgRNA or gRNA), which targets the Cas9 nuclease to the target genomic sequence and 3) a donor DNA template (e.g., single strand oligonucleotide (ssODN), long single strand DNA or double strand DNA (dsDNA) to direct homologous repair of the target locus (e.g., one associated with NOA, POI, or other genetic disorder). When introduced into cells (for example as part of a single vector or plasmid, or divided into multiple vectors or plasmids), the guide nucleic acid molecule guides the Cas9 to the target locus (e.g., one associated with NOA, POI, or other genetic disorder) and Cas9 will cut the target site. Cas9 unwinds the DNA duplex and cleaves one or both strands upon recognition of a target sequence by the guide nucleic acid molecule, but only if the correct protospacer-adjacent motif (PAM) is present at the 3′ end. Non-homologous end joining (NHEJ) repair of this cut will result in small insertions and deletions (indels), so the technique can be used to knockout genes. The technique can also be used to “re-write” the genetic sequence at the cut site through homology-directed repair (HDR). Using this system, DNA sequences within the endogenous genome and their functional outputs are easily edited or modulated.

As an alternative to expressing Cas9 via appropriate nucleic acid molecules, the guide nucleic acid molecule and Cas9 protein can also be delivered to the target cell (e.g., somatic cell of the testis (such as Sertoli cell, peritubular myoid cell, Leydig cell, or combinations thereof) or a SSC from the testis or patient derived iPScs) in fixed amounts using encapsulation techniques (e.g., using exosomes, liposomes, or both).

### 1. Introduction of Cas9 Protein Directly into a Target Cell

In one example, the Cas9 protein is expressed in a cell, such as E. coli, and purified. The resulting purified Cas9 protein, along with an appropriate guide nucleic acid molecule (sgRNA) specific for the target gene associated with NOA, POI, or other genetic disorder and donor DNA template (e.g., ssODN, long single strand DNA or dsDNA), are then introduced into a cell (e.g., somatic cell of the testis (such as Sertoli cell, peritubular myoid cell, Leydig cell, or combinations thereof) or a SSC from the testis or a male or female patient-derived iPSC) where gene expression can be regulated. In some examples, the Cas9 protein and guide nucleic acid molecule are introduced as separate components into the target cell. In other examples, the purified Cas9 protein is charged with the guide nucleic acid (e.g., gRNA), and this mixture is introduced into target cells (e.g., using transfection, transduction, or injection into the testis).

Once the Cas9 protein and guide RNA and donor DNA template are in the cell, one or more genetic defects associated with NOA or POI are corrected.

### 2. Introduction of Cas9 mRNA Directly into Target Cell

In one example, the Cas9 mRNA is introduced directly into the target cell (e.g., somatic cell of the testis (such as Sertoli cell, peritubular myoid cell, Leydig cell, or combinations thereof) or a SSC from the testis or a male or female patient-derived iPSC), along with an appropriate guide nucleic acid molecule (sgRNA) specific for the target gene associated with NOA, POI, or other genetic disorder, and donor DNA template (e.g., ssODN, long single strand DNA or dsDNA).

### 3. Expression of Cas9 from Nucleic Acids

In one example, the Cas9 protein is expressed from a nucleic acid molecule in a target cell (e.g., somatic cell of the testis (such as Sertoli cell, peritubular myoid cell, Leydig cell, or combinations thereof) or a SSC from the testis or a male or female patient-derived iPSC) containing a target gene whose genetic defect is to be corrected. In addition, these nucleic acid molecules are co-expressed in the cell/organism with the guide nucleic acid molecule (e.g., sgRNA) specific for the target whose genetic defect is to be corrected and a homologous donor DNA template.

In one example, multiple plasmids or vectors or proteins or nucleic acid molecules are used for the gene editing. The nucleic acid molecule encoding the Cas9 can be provided for example on one vector or plasmid, the guide nucleic acid molecule (e.g., gRNA) on another plasmid or vector, and the donor DNA template on another plasmid or vector. Multiple plasmids can be mixed and transfected into cells at the same time. But one skilled in the art will appreciate that other methods can be used to introduce these sequences, such as viral transduction using a lentivirus, adeno-associated virus (AAV), retrovirus, adenovirus, or alphavirus.

In some examples, multiple nucleic acid molecules are expressed from a single vector or plasmid. For example, a single plasmid can include the nucleic acid molecule encoding the Cas9 and the guide nucleic acid molecule. In some examples a plurality of different guide nucleic acid molecules (e.g., gRNAs), one for each target (such as 1, 2, 3, 4, 5, or 10 different targets), are present on a single plasmid or introduced separately. The donor DNA template (e.g., ssODN, long single strand DNA or dsDNA) is usually added as a separate nucleic acid molecule from the Cas9 (protein, mRNA or plasmid)

The nucleic acid molecules expressed in the target cell can be under the control of a promoter (such as UBC, EF1a, PGK, ROSA, Chicken β actin (CAG), CMV, H1, or U6) and contain selection markers (such as antibiotic resistance). Expression of different nucleic acid molecules may be driven by different promoters. For example, the U6 promoter may be used to drive expression of sgRNAs while EF1a or CAG promoters are used to drive expression of Cas9.

The resulting recombinant cell will express the Cas9 protein, along with the guide nucleic acid molecule specific for the target gene and the donor DNA template. Once the Cas9 protein is expressed, gene expression can be controlled in the target cell (e.g., somatic cell of the testis (such as Sertoli cell, peritubular myoid cell, Leydig cell, or combinations thereof) or a SSC from the testis or a male or female patient-derived iPSC).

V. Exemplary Target Genes Associated with NOA and POI

One or more genes can be targeted by the disclosed methods, such as at least 1, at least 2, at least 3, at least 4 or at least 5 different genes in the male with NOA (such as NOA-MA), such as 1, 2, 3, 4, 5, 6, 7, 8, 9 or 10 different genes. In one example, the gene is associated with a NOA or POI. Examples of target genes associated with NOA include, but are not limited to TEX11, TEX15, TAF4B, ZMYND15, SPINK2, NR5A1, SOHLH1, SYCE1, MCM8, androgen receptor (AR), AFF4 and AKAP9. Examples of target genes associated with POI include, but are not limited to MCM8, DCAF17 (C2orf37), FMR1.

Exemplary types of mutations include one or more nucleotide or amino acid deletions, substitutions, and insertions, or combinations thereof. In one example, the mutation associated with NOA includes a frameshift mutation. In one example, the mutation associated with NOA is an autosomal recessive mutation.

In one example, the subject has a testis-expressed 11 gene (TEX11) mutation, such as a V748A mutation, and those disclosed in Yang et al. (EMBO Molecular Med 7:1198-1210, 2015) and Yatsenko et al. (NEJM 372:2097-2107, 2015), both herein incorporated by reference.

In one example, the subject has a testis-expressed 15 gene (TEX15) mutation, such as a nonsense mutation or a single nucleotide deletion leading to premature stop codons (c.2419A>T, p.Lys807*, and c.3040delT, p.Ser1014Leufs*5, respectively).

In one example, the subject has an androgen receptor (AR) mutation, such as a Gln798Glu and/or R630W substitution, or a CAG repeat in exon 1.

In one example, the subject has a mutation in TAF4B or ZMYND15(see Ayhan et al., J. Med. Genetics 41:239-44, 2014, which discloses mutations in other genes as well, herein incorporated by reference).

In one example, the genetic defect associated with NOA is decreased expression of a gene or gene product, such as AR, wherein the disclosed methods can be used to express native (i.e., wild-type, non-mutated) AR, thereby increasing expression of the desired protein.

Other exemplary mutations that can be corrected with the disclosed methods are provided in Table 1.

TABLE 1
Exemplary Genetic mutations associated with NOA or POI

| | Mutation (nucleic acid or protein) |
|---|---|
| Gene Associated with NOA | |
| SOHLH1 | c.346-1G > A |
| TEX11 | c.792+1G −> A |
| | protein: V748A |
| GCNA | c.1323G > T |
| PORCN | c.1099C > T |
| MAGEB10 | c.982G > A |
| AKAP4 | c.G241A |
| FMR1 | c.T522A |
| SCML2 | c.40_42del |
| | c.T2095C |
| SOX3 | c.G14A |
| | c.G157C |
| | c.C307A |
| TEX15 | c.2419A > T, p.Lys807* |
| | c.3040delT, p.Ser1014Leufs*5 |
| TAF4B | p.R611X (see Ayhan et al., J Med |
| | Genet, 2014. 51(4): 239-44) |
| ZMYND15 | p.K507Sfs*3 (see Ayhan et al., J Med |
| | Genet, 2014. 51(4): 239-44) |
| SPINK2 | c.56-3C > G (see Kherraf et al., EMBO |
| | Mol Med, 2017. 9(8): 1132-1149) |
| NR5A1 | p.Gly123Ala (c.368G > C) |
| | p.Pro129Leu (c.386C > T) (see |
| | Bashamboo et al., Am J Hum Genet, |
| | 2010. 87(4): 505-12) |
| SYCE1 | c.197-2 A > G |
| MCM8 | c. 1954-1 G > A |
| androgen receptor (AR) | c.646G > A |
| AFF4 | c.3319A > G; c.1048 > G |
| AKAP9 | c.C1826A; C.A5680G |
| Gene Associated with POI | |
| MCM8 | C.446OG |
| | c.1469-1470insTA |
| DCAF17 (C2orf37) | c.127-1G > C; c.535C > T |
| FMR1 | c.T522A |

### VI. Exemplary Target Cells

Also provided are recombinant or transformed somatic cells of the testis (such as Sertoli cell, peritubular myoid cell, or Leydig cell), recombinant/transformed SSCs from the testis, and recombinant/transformed male of female patient-derived iPSCs, which express the recombinant nucleic acid molecule used to correct a genetic defect associated with NOA, POI, or other genetic disorder.

In some examples the target cell is an iPSC obtained from a male subject with NOA or a female subject with POI, (or male or female with another genetic disorder) which can be transformed/transfected with the recombinant nucleic acid molecule that can correct a genetic defect associated with NOA, POI, or other genetic disorder, differentiated to PGCLCs that can be transplanted into the testes or ovaries or haploid eggs or sperm that can be used to produce human embryos in an in vitro fertilization (IVF) laboratory.

### VII. Treatment of Female Infertility

In some examples, methods similar to those described for treating NOA are used to treat female infertility. In such examples, instead of the recombinant nucleic acid molecule correcting a genetic defect associated with NOA males (for example at the pluripotent iPSC stage), it corrects a defect associated with POI in females. In addition, in such examples, iPSCs obtained from the female subject can be genetically corrected using a recombinant nucleic acid molecule that can correct a genetic defect associated with female infertility, differentiated to primordial germ cell-like cells (PGCLCs) that can be transplanted into the ovary or eggs that can be fertilized ex vivo.

In addition, methods for treating NOA can utilize iPSCs, for example instead of SCCs. For example, iPSCs obtained from the male subject with NOA are genetically corrected using a recombinant nucleic acid molecule that can correct a genetic defect associated with NOA (for example at the pluripotent iPSC stage), differentiated to PGCLCs, which can be transplanted into the testis or differentiated to sperm that can be used to fertilize eggs ex vivo.

Hayashi et al. (Cell, 2011. 146(4):519-32) and Hayashi et al. (Science, 2012. 338(6109):971-975) describe derivation of transplantable PGCLCs for male and female resulting in birth of live mice. Zhou (Cell Stem Cell, 2016. 18(3):330-40) and Hikabe et al. (Nature, 2016. 539(7628):299-303) describe differentiation from iPSCs or ESCs to PGCLCs and then on to eggs or sperm, completely in vitro.

### EXAMPLE 1

### In Vivo Sertoli Cell Gene Therapy

For mutations in Sertoli cells that cause NOA-MA, gene therapy vectors can be injected directly into the seminiferous tubules of the testis, via the efferent ducts, as previously described⁴⁵. Gene therapy vectors can be introduced into Sertoli cells using viruses (e.g., adenovirus, adeno-associated virus, or lentivirus); electroporation or transfection reagents (e.g., lipofectamine, polyethyleneimine (PEI), etc.). In 2002, three groups independently demonstrated that in vivo Sertoli cell gene therapy could reverse the infertile phenotype in “Steel” mice that lack the Kit Ligand in Sertoli cells and exhibit and NOA-MA phenotype. The three studies used adenovirus, lentivirus and electroporation, respectively, and sperm production was restored in treated males⁴⁶⁻⁴⁸. Offspring were produced in two of those studies and there was no evidence that the gene therapy vector was transmitted to resulting progen^{y46,48.}

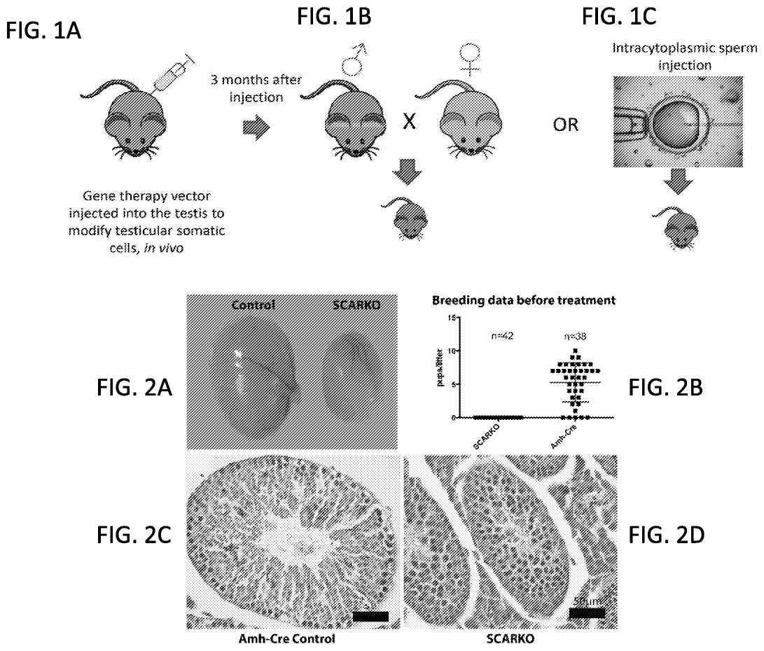

In humans, mutations in the Kit signaling pathway lead to the Piebald condition⁴⁹, which is characterized by patches of pale hair or skin, but association with infertility by linkage analysis is not strong⁵⁰. Review of the gnomAD sequencing database of 120,000 controls sequenced by whole-genome sequencing (gnomad.broadinstitute.org/gene/ENSG00000049130) reveals 0 biallelic knockout individuals in the cohort. However, other human Sertoli cell gene variants are associated with azoospermia and reproduce an NOA phenotype when modeled in mice (e.g., AR, AFF4, AKAP9)^{18-21,51-55}. Sertoli cell gene therapy approaches can be used to restore fertility in mouse models of human NOA, such as androgen receptor (AR) mutations (FIG. 1). in vivo Sertoli cell gene therapy reversed the infertile phenotype in Sertoli cell androgen receptor knockout (SCARKO) mice. SCARKO mice have small testes and are infertile due to arrested spermatogenic development at the spermatocyte stage (FIGS. 2A-2D).

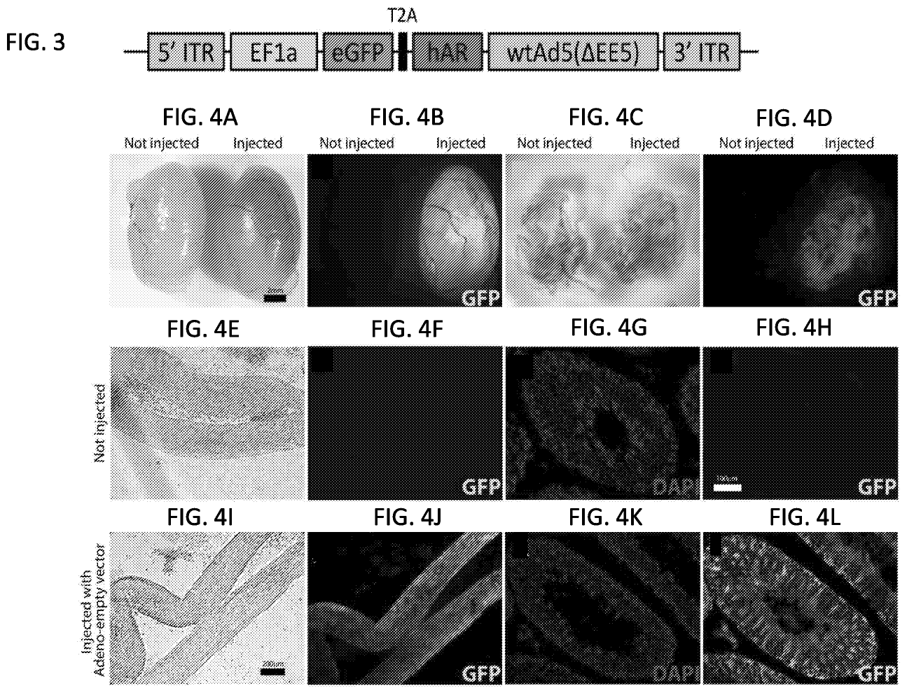

An adenovirus vector was designed to express the enhanced green fluorescent protein (eGFP) and a therapeutic human androgen receptor (hAR) gene under the control of the elongation factor 1a (EF1a) promoter (FIG. 3). The Ad-EF1a-eGFP-hAR gene therapy vector was injected via the efferent ducts into the seminiferous tubules of infertile SCARKO mice. Testes were collected from control (not injected) and Ad-EF1a-eGFP-hAR treated animals one week after injection. Expression of the eGFP reporter gene indicates that Sertoli cells along the length of the seminiferous tubules were efficiently transduced with the Ad-EF1a-eGFP-hAR gene therapy vector (FIGS. 4A-4L).

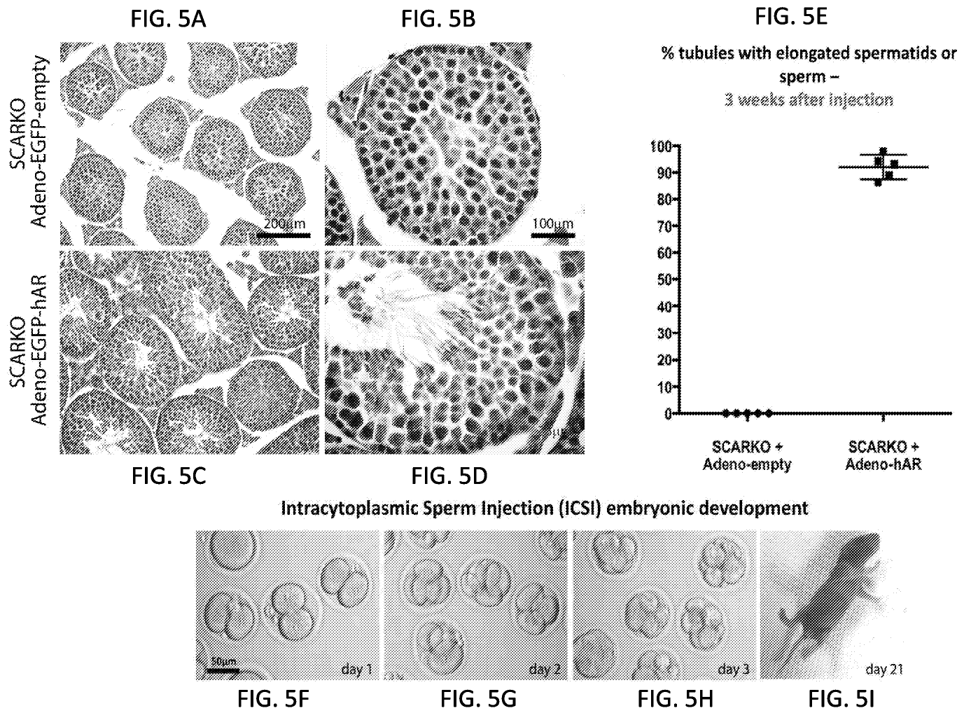

The histology of Ad treated SCARKO mice was examined 3 weeks after injection. Compared with SKARKO mice treated with the empty vector Ad-EF1a-eGFP-Empty, which exhibited an NOA phenotype with maturation arrest (FIGS. 5A-5B), spermatogenesis was restored in 90% of seminiferous tubules of mice treated with the Ad-EF1a-eGFP-hAR vector (FIGS. 5C-5E). Also, all seminiferous tubule lumens were open indicating that the treatment restored fluid secretion by Sertoli cells (FIG. 5D). Sperm recovered from the cauda epididymis of Ad-EF1a-eGFP-hAR treated SCARKO mice were competent to fertilize mouse eggs by intracytoplasmic sperm injection (ICSI), leading to preimplantation embryo development (FIGS. 5F-5H) and production of live born offspring (FIG. 5I).

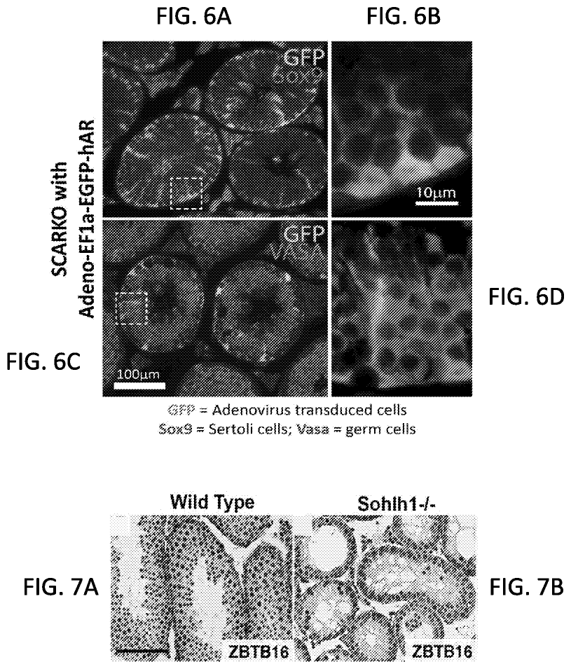

Immunohistochemical co-staining for eGFP (gene therapy vector) and SOX9 (Sertoli cell marker) or VASA (germ cell marker) revealed that the Ad-EF1a-eGFP-hAR vector efficiently transduced Sertoli cells (FIGS. 6A-6B), but not germ cells (FIGS. 6C-6D). To further establish the absence of germline modification/germline transmission, adenovirus treated males were bred continuously with wild type females to produce 259 progeny. None of the progeny carried the EF1a-eGFP-hAR transgene.

### EXAMPLE 2

Ex Vivo Gene Therapy and Transplantation of Transformed Male Germline Stem Cells

Prior gene therapy studies produced transgenic rodent models for basic research where germline transmission was desired⁵⁶⁻⁶⁰. For example, Wu and colleagues corrected a genetic disease (cataracts) using this approach, but germline transmission was the desired outcome⁶⁰. However, there are societal concerns about germline gene therapy if it involves transmission to subsequent generations. This example provides a new approach to germline gene therapy using spermatogonial stem cells (SSCs) without germline transmission.

SOHLH1 and TEX11 are examples of autosomal recessive and X-linked recessive mutations, respectively, that cause NOA in mice and men¹³⁻¹⁶. Men may be particularly susceptible to X-linked recessive diseases because they have only one X chromosome and there is an abundance of spermatogonial genes on the X chromosome⁶¹. Three approaches to precision gene therapy by ex vivo modification and transplantation of spermatogonial stem cells (SSCs) (germline gene therapy) are described (two for SOHLH1 mutations and one for TEX11 mutations). SSC can be modified using any of a variety of transfection/transduction reagents. However, in the examples provided here, the use of polyethyleneimine (PEI) is described.

The sgRNA sequences used in this Example are shown in Table 2:

TABLE 2
sgRNA sequences

| | |
|---|---|
| Top strand (SEQ ID NO:) | Bottom strand (SEQ ID NO:) |

sgRNAs for gene therapy in Sohlh1-KO mouse model

| | | |
|---|---|---|
| sgLHA-PGK 92-1 | CACCGCGAAGCCCATCGAATTCTA | AAACGTAGAATTCGATGGGCTTC |
| | C (1) | GC (2) |
| sgLHA-PGK 92-2 | CACCGTAGAATTCGATGGGCTTCG | AAACGCGAAGCCCATCGAATTCT |
| | C (3) | AC (4) |
| sgSohlh1-3 new | CACCGCGGGCAACACTTGCCCCCT | AAACTAGGGGGCAAGTGTTGCCC |
| | A (5) | GC (6) |
| sgSohlh1-4 | CACCGCCGTATGTGATGCCAGTGT | AAACTACACTGGCATCACATACG |
| | A (7) | GC (8) |

sgRNAs for gene therapy in Tex11-KO mouse model

| | | |
|---|---|---|
| sgTEX11-1 | CACCGGCTGCAACGGCTGCCCTTT | AAACAAAAGGGCAGCCGTTGCAG |
| | T (9) | CC (10) |
| sgTEX11-2 | CACCGGCAGCAACCAGTTCATCTC | AAACCGAGATGAACTGGTTGCTG |
| | G (11) | CC (12) |

sgRNAs targeting Rosa26 locus for safe harbor gene therapy

| | | |
|---|---|---|
| sgRosa26-1 YS | CACCGGGCAGGCTTAAAGGCTAA | AAACGGTTAGCCTTTAAGCCTGC |
| | CC (13) | CC (14) |
| sgRosa26-2 YS | CACCGGTCCTGCAGGGGAATTGAA | AAACGTTCAATTCCCCTGCAGGA |
| | C (15) | CC (16) |

These sgRNAs (SEQ ID NOS: 1-16) can be cloned into the BbsI restriction site of the plasmid shown in SEQ ID NO: 17. This plasmid is derived from pSpCas9(BB)-2A-GFP (pX458) (addgene #48138) and pEF-ENTR A (696-6) (addgene #17427). This plasmid contains the site for cloning sgRNAs, and CMV-driven Cas9-EGFP from pX458 and the backbone of p696-6. The sgRNAs can be driven by the U6 promoter.

Exemplary donor template sequences are provided in SEQ ID NOS: 18-22 (pUC19 Donor Soh1h1 mCherry PURO-1 SEQ ID NO: 18; pUC19 Donor Soh1h1 mCherry PURO-2 SEQ ID NO: 19; pUC19 Donor mCherry TEX11 SEQ ID NO: 20; pUC19 Donor Rosa26 PGK-puromycin-T2A-mCherry-T2A-Sohhlhl cDNA-sv40polyA SEQ ID NO: 21; pUC19 Donor Rosa26 PGK-puromycin-T2A-mCherry-T2A-Tex11 cDNA-sv40polyA SEQ ID NO: 22). The backbones of these donor templates are pUC19.

Soh1h1−/31 mice are infertile with an NOA-MA phenotype (FIGS. 7A-7B). Wild type mice have ZBTB16+ cells (marker of stem and progenitor spermatogonia) on the basement membrane of seminiferous tubules; multiple layers of germ cells and open lumens of the seminiferous tubules (FIG. 7A). In contrast, Soh1h1−/− mice are infertile with an early maturation arrest phenotype. Soh1h1−/− mice have the ZBTB16+ stem and progenitor spermatogonia in the seminiferous tubules but these cells are unable to differentiate and produce sperm (FIG. 7B).

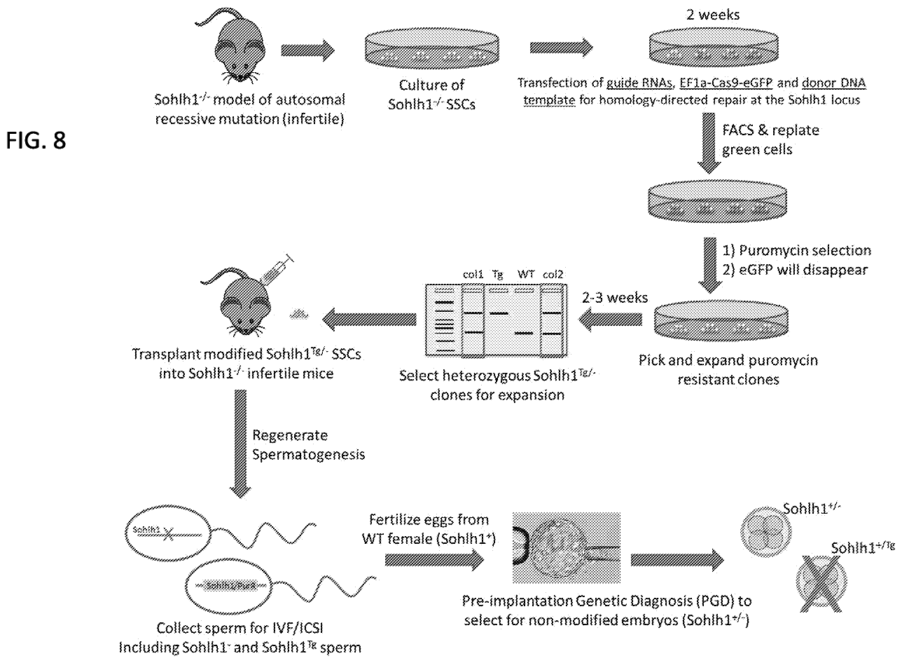

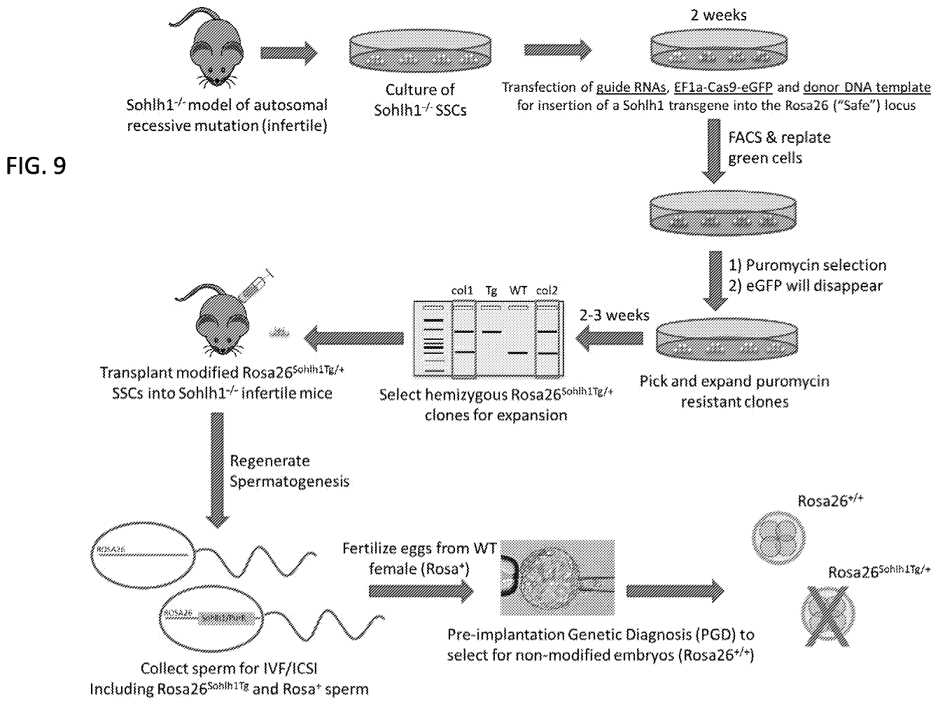

A functional Soh1h1 gene can be introduced into the ZBTB16+ spermatogonia extracted from Soh1h1−/−testes, ex vivo, and the modified cells can be transplanted to regenerate complete spermatogenesis. FIGS. 8 and 9 describe how CRISPR/Cas9 can be used to insert a functional SOHLH1 gene (cDNA) into the endogenous (non-functional) SOHLH1 locus or the “safe harbor” ROSA locus of SOHLH1−/− mice. The ROSA locus is permissive to therapeutic gene expression and insertion into this locus is not known to cause adverse outcomes.

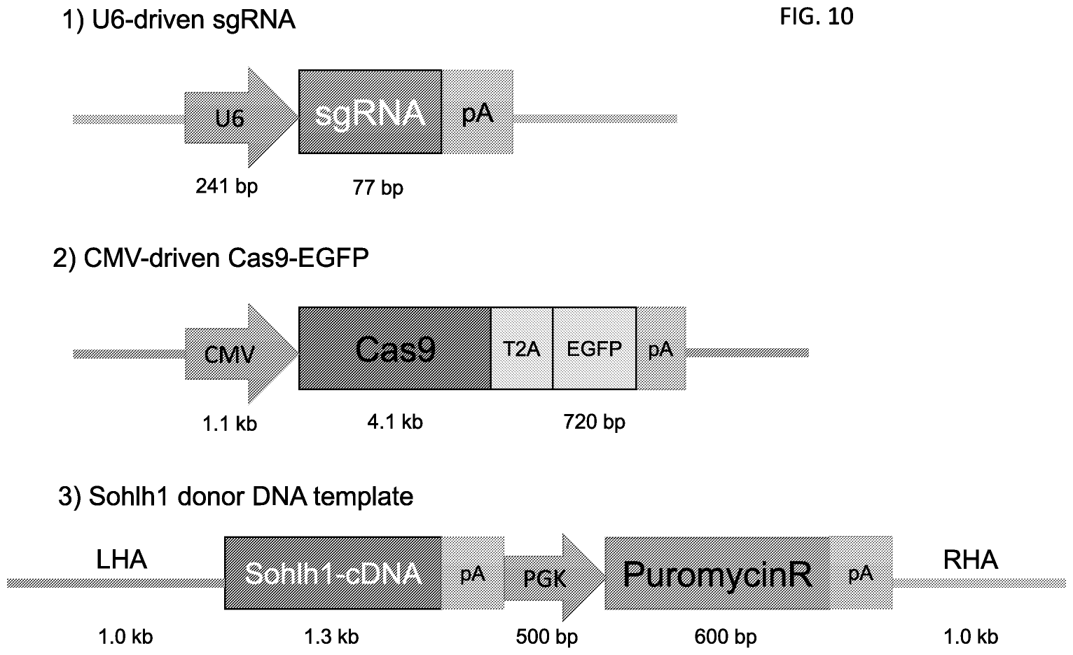

Insertion of Soh1h1 cDNA into the endogenous Soh1h1 locus. FIG. 8 describes insertion of a functional Soh1h1 cDNA plus a puromycin resistance cassette into the endogenous (mutant) Soh1h1 locus, immediately downstream of the endogenous Soh1h1 promoter. First, spermatogonial stem cells (SSCs) are isolated from the testes of Soh1h1−/− mice and cultured ex vivo. Once cultures are established, polyethyleneimine (PEI) or other transfection/transduction reagents are used to introduce 1) U6 promoter driven guide RNAs (sgRNAs) targeted immediately downstream of the endogenous Soh1h1 promoter; 2) a plasmid containing a CMV promoter driven bicistronic Cas9-eGFP transgene; and 3) a donor DNA template featuring a promoterless Soh1h1 cDNA and a PGK driven puromycin resistance (PurR) gene flanked by left and right homology arms into the cultured SSCs (FIG. 10). The CRISPR sgRNAs are designed to target Cas9 cutting immediately downstream of the endogenous Soh1h1 or Rosa promoters such that those promoters will drive expression of the Soh1h1 cDNA. With this design, the CMV-Cas9-eGFP transgene is expressed transiently while the Soh1h1-PGK-PurR sequence is inserted into the host cell genome and stably expressed. Transduced cells are then identified by expression of an eGFP reporter gene, selected by FACS, and retured to culture. Cultured cells are then treated with puromycin to select for cells with stable expression of the puromycin resistance (PurR) gene. After puromycin selection, surviving germ cell clusters are picked, expanded clonally and genotyped to identify heterozygous clones that have the corrective transgene on only one allele (Soh1h1^{Tg/-})(FIG. 8). Correctly modified clones are further expanded ex vivo and the Soh1h1^{tg/-} SSCs are transplanted into the testes of Soh1h1^{-/-} recipients. SSCs with one functional copy of the Soh1h1 gene will regenerate spermatogenesis and produce functional sperm. Half of the resulting sperm will have the mutant Soh1h1 allele (−) and half will have the corrected allele (tg). When the resulting sperm are used to fertilize eggs from WT female mice (e.g., using ICSI), half of the embryos will contain the corrective transgene (Soh1h1^{+/Tg}) from Dad and half will contain the mutant transgene from Dad (Soh1h1^{+/-}). All embryos will contain a healthy Soh1h1 transgene (+) from Mom. Preimplantation genetic diagnosis (PGD) can then be used to select only the transgene-free heterozygous embryos for transfer (Soh1h1^{+/-}). With this design, all F1 progeny will be carriers of the mutant Soh1h1 allele from Dad, but fertile because they will inherit a healthy Soh1h1 allele from Mom. The mutant allele will be further diluted with each successive generation (F2: 50% of offspring will be carriers; F3: 25% will be carriers; F4: 12.5% will be carriers, etc.), assuming that partners always introduce a healthy Soh1h1 allele. Therefore, the disclosed methods can be used to treat a man for his genetic infertility by germline gene therapy and, without passing genetic modification to his progeny, remove infertility susceptibility from his entire family lineage.

Inserting the corrective transgene into the endogenous locus will allow regulation of expression from the endogenous promoters, as the CRISPR/Cas9 technology enables precise genomic integration. SSC culture ex vivo allows selection and expansion of only the accurately modified SSC clones. In some examples, random genomic integration is avoided, as it has led to disease and death in previous gene therapy trials^{71,72}. An alternative to inserting the therapeutic transgene into the endogenous locus is insertion into a “safe harbor” location in the genome such as the ROSA26 locus.

Inserting the Soh1h1 cDNA into the Rosa26 “Safe Harbor” locus. FIG. 9 provides a schematic approach for inserting the therapeutic transgene into the Rosa26 locus. The approach is similar to insertion into the endogenous locus (FIG. 8), except SSC clones are selected that are hemizygous at the ROSA locus and homozygous null at the Soh1h1 locus (ROSA^{Tg/+}; Soh1h1^{-/-}). After transplantation of correctly modified SSCs, the resulting haploid sperm bear the genotypes Rosa26^{Tg}/Soh1h1⁻ or Rosa26⁻/Soh1h1⁻. Eggs from a WT female are fertilized, which will have two healthy Soh1h1 alleles (Soh1h1^{+/+}). Half of the resulting progeny have the genotype Soh1h1^{+/-}/Rosa26^{+/+} and half will have the genotype Soh1h1^{+/-}/ROSA^{Tg}. Pre-implantation genetic diagnosis (PGD) can be used to identify the Soh1h1^{+/-}/Rosa26^{+/-} embryos ex vivo for subsequent transfer into pseudopregnant females. Similar to the situation with insertion into the endogenous locus, progeny will be heterozygous at the Soh1h1 locus (Soh1h1^{+/-}) and fertile. Again, the mutant Soh1h1 allele should be further diluted in each successive generation, assuming partners introduce functional copies of the Soh1h1 gene.

Treating X-linked recessive disorders by ex vivo gene therapy followed by transplantation of SSCs in men with NOA. Also provided are methods for treating X-linked recessive disorders (such as those associated with mutations in Tex11). Males have only a single X chromosome. Other exemplary X-linked infertility-associated genes include GCNA, PORCN, MAGEB10, AKAP4, FMR1, SCML2, and SOX3. Thus, mutations in these genes can also be corrected with the disclosed methods.

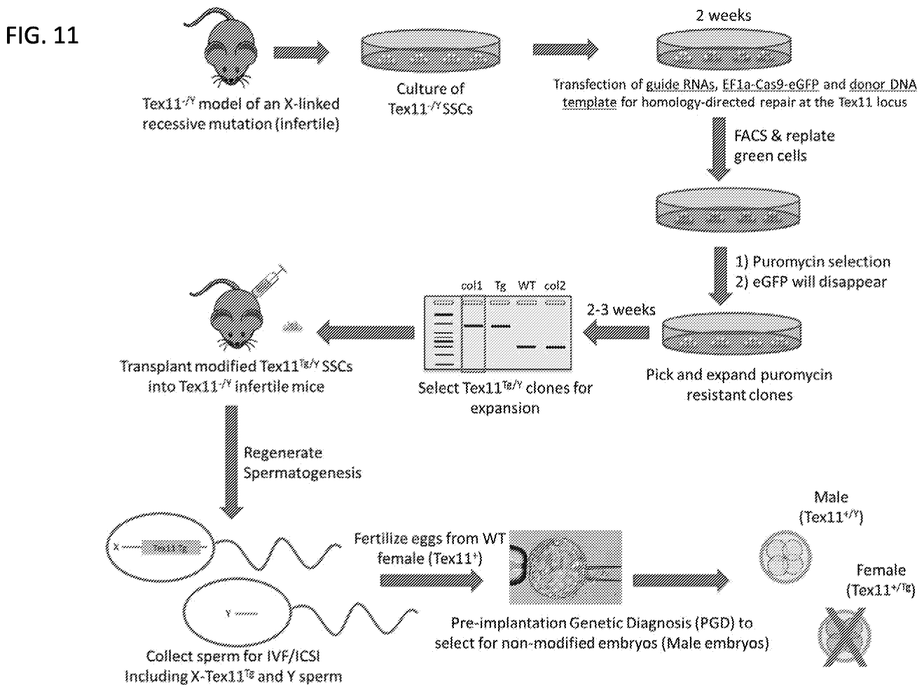

The therapeutic transgene can be targeted to the endogenous locus on the X chromosome or to a safe harbor locus, such as ROSA. The approach for targeting the ROSA locus is similar to what is shown in FIG. 9. The approach for targeting the endogenous locus on the X chromosome is described in FIG. 11. SSC clones are selected with the Tex11^{Tg/Y} genotype. When the appropriately modified SSCs are transplanted, they will regenerate sperm with the genotypes Tex11^{Tg} or Y. Sperm are then used to fertilize WT eggs, which have a healthy copy of the Tex 11 gene (Tex11⁺). The resulting male embryos will have the genotype Tex11^{+/Y} and female embryos will have the genotype Tex11^{+/Tg}. Male embryos are selected for transfer to pseudopregnant females (e.g., using PGD). Male embryos will not have the corrective transgene because they received the unmanipulated Y chromosome (not the modified X chromosome) from Dad. In this scenario, the pathogenic allele is eliminated from the family lineage in the first generation. The precedent for male chromosome sex selection is established to prevent germline transmission after mitochondrial replacement therapy⁷³. Mitochondria are inherited from Mom, not Dad. Therefore, selection of male embryos prevents germline transmission of donor mitochondria.

Ex vivo gene therapy followed by transplantation of SSCs to correct infertility AND genetically linked comorbid diseases. Mutations in MCM8 are associated with NOA in men and POI in women and produce similar infertile phenotypes when modeled in mice. Mutations in the MCM8 locus are also associated with DNA damage repair defects and cancer with an autosomal recessive pattern of inheritance. Therefore, treating MCM8 mutations in men with NOA using ex vivo gene therapy followed by transplantation of SSC as described in FIG. 8 Soh1h1 mutations, can removing both infertility and DNA damage/repair and cancer susceptibility defects from the entire family lineage. As described above, this can be achieved without passing the therapeutic transgene from DAD to progeny because Mom will contribute a healthy MCM8 allele. Other exemplary genes associated with infertility and comorbid disease phenotypes are FMR1 (male and female infertility; mental retardation), DCAF17 (male and female infertility; Woodhouse-Sakati syndrome characterized by diabetes, alopecia, neurological problems and other phenotypes).

### EXAMPLE 3

### Ex Vivo Gene Therapy in Male or Female Patient-Derived iPSCs Followed by Differentiation to Transplantable PGCLCs or Eggs or Sperm

As oogenesis is not a stem cell-based system, this example describes methods to treat infertility and comorbid diseases in men and women using patient-derived iPSCs.

Males or females with mutations that cause germ cell development defects leading to NOA or POI can be treated by ex vivo gene therapy in patient-derived iPSCs followed by differentiation to PGCLCs that can be transplanted into the ovaries or testes or eggs or sperm that can be used to produce embryos in the IVF clinic. Exemplary genetic mutations that cause NOA in males, POI in females and reproduce similar infertility phenotypes when modeled in mice are listed above (e.g., MCM8, FMR1, and DCAF17). Mutations in these genes and the associated phenotypes have a recessive mode of inheritance and are therefore amenable to germline gene therapy without germline transmission approach described above. Selection and expansion of heterozygous or hemizygous male or female patient-derived iPSC clones as described for SSC clones in FIGS. 8 and 9 can lead to the production of both heterozygous/hemizygous and transgenic embryos. Selection of homozygous/hemizygous embryos lacking the transgenic modifications will produce offspring that are carriers of the pathogenic mutation, but fertile. As described above, the pathogenic mutation will be diluted in each successive generation until it is essentially eliminated from the family lineage. If the pathogenic infertility associated mutation is also associated with a comorbid disease (e.g., MCM8, FMR1, or DCAF17), then the comorbid disease will also be eliminated from the family lineage.

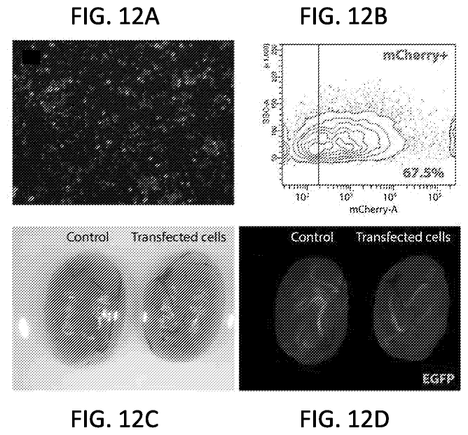

PEI was used as the transfection reagent to introduce therapeutic transgenes into cultured SSCs. PEI is a cationic chemical reagent used to transiently transfect mammalian cells but has not been used in SSCs⁷⁴⁻⁷⁶. The published PEI transient transfection protocols were modified to be compatible with cultured mouse SSCs by replacing saline or OptiMEM with Iscove's Modified Dulbecco's Medium (IMDM) culture medium. Saline and OtiMEM were toxic to SSCs. SSCs are slowly cycling cells and genetic modification with most transfection/transduction reagents is very inefficient. The most efficient transduction protocols in mice have been with lentiviral vectors that have mechanisms to cross the cell and nuclear membranes and integrate their genomes into the chromosomes of non-dividing cells^{58,77}. Lentiviral vectors are much less effective for transducing SSCs in nonhuman primates⁷⁸. Thus, it is not obvious that a cationic reagent like PEI would lead to efficient transduction of SSCs. However, the data in FIG. 12A-B demonstrate that nearly 70% of cultured mouse SSCs can be transfected with a vector carrying the mCherry reporter gene using the PEI transfection reagent. Furthermore, the mCHERRY+transfected cells could be transplanted into infertile recipient testes where they produced colonies of spermatogenesis (visualized by GFP fluorescence in all cells) that were quantitatively and qualitatively similar to untransfected SSCs (FIG. 12D).

### EXAMPLE 4

### Validation of sgRNAs Targeting Human

### SOHLH1 and TEX11 Sequences Associated with NOA

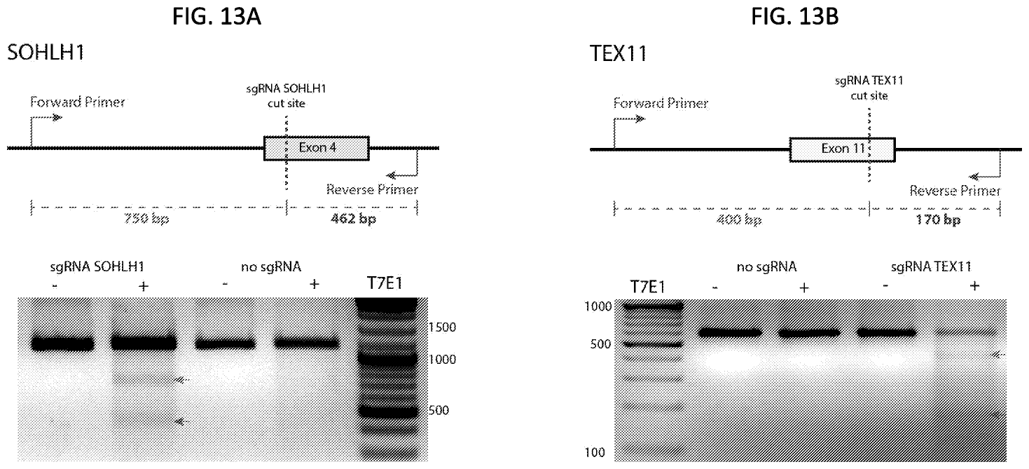

To enable CRISPR/Cas9 gene editing technologies, guide RNAs targeting the pathogenic alleles are used. FIGS. 13A and 13B provide T7E1 validation assay (Innovative Genomics) data for sgRNAs targeting Exon 4 of the human SOHLH1 gene and Exon 11 of the human TEX11 gene.

T7 endonuclease cleaves double-stranded DNA at positions of mismatches. Nonhomologous end joining (NHEJ) repair of CRISPR/Cas9-induced breaks will leave a variety of different mutations, and there will almost always be some wild type sequence remaining. Thus, when you amplify the target region, denature and renature the products, there will be mismatches at the target site, if cleavage was effective.

The sgRNA for SOHLH1 was designed to target a region containing a c.346-1G>A mutation, which was identified in an NOA patient⁷⁹. The mutation resulted in partial deletion at a cryptic splice site within exon 4, which leads to truncated bHLH domain. The sequence of sgRNA targeting this region was ATTTCAGATTCTTGCTTCCT (SEQ ID NO: 23), which targets within 10 bp-range of the mutation. 293AD cells were transfected with plasmid DNA (sgRNA-Cas9 plasmid) containing sgRNA and Cas9 sequences using PEI (50 μg/mL with 2 μg of plasmid DNA). Cells were collected 72 hours later. The target locus was PCR amplified and digested with the T7 endonuclease. When the products were run on a gel, a pattern that includes a band representing the undigested wild type sequences as well as the two smaller digestion products of expected sizes (750 base pairs and 462 base pairs) indicates accurate Cas9 cutting of the target SOHLH1 locus (FIG. 13A).

The sgRNA targeting TEX11 was designed to target a region containing a c.792+1G->A mutation, which was identified in a human NOA patient by Yatsenko et al.¹⁵. The mutation is located at the splicing donor site of TEX11 intron 11. The sequence for the sgRNA was CTGGGCCAGAAATGCTGGTA (SEQ ID NO: 24), targeting within 10 bp-range of the mutation. 293AD cells were transfected with the plasmid DNA containing sgRNA and Cas9 sequences. Cells were collected 72 hours later. The target locus was PCR amplified and digested with the T7 endonuclease. Digestion products were run on a gel and revealed bands consistent with the undigested wilt type sequences as well as two smaller digestion products of expected size (400 bp and 170 bp).

Below are the sequences for the top and bottom strands of each sgRNA shown in SEQ ID NOS: 23 and 24. These sequences target the mutated region in wild-type cells and can be used to clone into the sgRNA-Cas9 plasmid. Thus, SEQ ID NO: 24 can be used to target the mutation in TEX11 by replacing nt G18 with “A” (since the mutation is c.792+1G>A). In addition, SEQ ID NO: 23 can be used to target both mutant and WT versions of SOHLH1 in the human genome.

Both sgRNA can be cloned into the sgRNA-Cas9 plasmid, since they target different genes (the sgTEX11 targets TEX11 and sgSOHLH1 targets SOHLH1). This will be corresponding with the last figure.

| | Top strand (SEQ ID NO:) | Bottom strand (SEQ ID NO:) |
|---|---|---|
| sgTEX11 Ex11 | CACCGCTGGGCCAGAAA | AAACTACCAGCATTTCTGGCCC |
| (intron12) 63-1 | TGCTGGTA (25) | AGC (26) |
| sgSOHLH1 Ex4 | CACCGCAACGAGTGCCA | AAACAGGAAATGTGGCACTCGT |
| (intron3)-75 | CATTTCCT (27) | TGC (28) |

### REFERENCES

1. Lee et al., Urology 2011;77:598-601.
2. Weedin et al., The Journal of Urology 2011;186:621-626.
3. Gudeloglu A, Parekattil S J. Update in the evaluation of the azoospermic male. Clinics (Sao Paulo, Brazil) 2013;68 Suppl 1:27-34.
4. Kim E et al., The Journal of urology 1997;157:144-146.
5. Hayashi et al., Cell 2011;146:519-532.
6. Hayashi et al., Science 2012;338:971-975.
7. Hikabe et al., Nature 2016;539:299-303.
8. Zhou et al., Cell Stem Cell 2016;18:330-340.
9. Dominguez et al., Scientific reports 2014;4:6432.
10. Ramatha et al., Scientific reports 2015;5:15041.
11. Irie et al., Cell 2015;160:253-268.
12. Park et al., Stem Cells 2009;27:783-795.
13. Ballow et al., Developmental Biology 2006;294:161-167.
14. Song et al., European Journal of Obstetrics & Gynecology and Reproductive Biology 2015;184:48-52.
15. Yatsenko et al., New Engl J Med 2015;372:2097-2107.
16. Yang et al.,. EMBO Molecular Medicine 2015;7:1198-1210.
17. Yang et al., Gene Dev 2008;22:682-691.
18. Goglia et al., Fertility and Sterility 2011;96:1165-1169.
19. Mirfakhraie et al., Journal of Andrology 2011;32:367-370.
20. Massin et al., Clinical endocrinology 2012;77:593-598.
21. Chen et al., Asian journal of andrology 2015;17:857-858.
22. Tenenbaum-Rakover et al., Journal of Medical Genetics 2015;52:391.
23. Gurbuz et al., Clin Genet 2018;93:853-859.
24. Utine et al., European journal of obstetrics, gynecology, and reproductive biology 2018;221:76-80.
25. National Academies of Sciences E, Medicine. International summit on human gene editing: A global discussion. Washington, DC: The National Academies Press; 2015.
26. National Academies of Sciences E, and Medicine. Human genome editing: Science, ethics, and governance. Washington, DC: The National Academies Press; 2017.
27. Eisenberg et al., Human Reproduction 2011;26:3479-3485.
28. Eisenberg et al.,Human Reproduction 2014;29:1567-1574.
29. Ventimiglia et al.,. Fertil Steril 2015;104:48-55.
30. Glaze et al., Multiple Sclerosis Journal 2017;0:1352458517734069.
31. Walsh et al., Cancer 2010;116:2140-2147.
32. Eisenberg et al., The Journal of Urology 2015;193:1596-1601.
33. Eisenberg et al., Fertility and Sterility 2015;103:66-71.
34. Walsh et al., Archives of Internal Medicine 2009;169:351-356.
35. Salonia et al., European Urology 2009;56:1025-1032.
36. Glazer et al., Semin Reprod Med 2017;35:282-290.
37. Ali et al., Clinical Genetics 2016;90:263-269.
38. Steindl et al., Clinical Genetics 2010;78:594-597.
39. Ben-Omran et al., Am J Med Genet A 2011;155a:2647-2653.
40. Alazami et al., The American Journal of Human Genetics 2008;83:684-691.
41. He et al., Oncogene 2017;36:3629-3639.
42. Lee et al., Nat Commun 2015;6:7744.
43. Lutzmann et al., Molecular Cell 2012;47:523-534.
44. Park et al., Molecular and Cellular Biology 2013;33:1632-1644.
45. Ogawa et al., Internatl J. Devel. Biol. 1997;41:111-122.
46. Ikawa et al., Proc Natl Acad Sci U S A 2002;99:7524-7529.
47. Yomogida et al., Biol Reprod 2002;67:712-717.
48. Kanatsu-Shinohara et al., Proc Natl Acad Sci U S A 2002;99:1383-1388.
49. Spritz R A. The Journal of investigative dermatology 1994;103:137S-140S.
50. Galan et al.,Human Reproduction 2006;21:3185-3192.
51. Meng et al., Biology of Reproduction 2011;85:254-260.
52. Chakraborty et al., Molecular Endocrinology 2014;28:1055-1072.
53. Matzuk M M, Lamb D J. Nature medicine 2008;14:1197-1213.
54. Schimenti et al., Genetics 2013;194:447-457.
55. Urano et al., Molecular and Cellular Biology 2005;25:6834-6845.
56. Orwig et al., Biol Reprod 2002;67:874-879.
57. Ryu et al., Dev Bio! 2003;263:253-263.
58. Ryu et al., Journal of Andrology 2007;28:353-360.
59. Sato et al., Stem Cell Reports 2015;5:75-82.
60. Wu et al., Cell research 2015;25:67-79.
61. Wang et al., Nat Genet 2001;27:422-426.
62. Brinster R L, Zimmermann J W. PNAS 1994;91:11298-11302.
63. Brinster R L, Avarbock M R. PNAS 1994;91:11303-11307.
64. Hermann et al., Cell Stem Cell 2012;11:715-726.
65. Honaramooz et al., Biology of Reproduction 2003;69:1260-1264.
66. Mikkola et al., Reproduction in Domestic Animals 2006;41:124-128.
67. Kim et al., Reproduction 2008;136:823-831.
68. Herrid et al., Biology of Reproduction 2009;81:898-905.
69. Izadyar et al., Biology of Reproduction 2003;68:272-281.
70. Richardson et al., PLoS ONE 2009;4:0006308.
71. Hacein-Bey-Abina et al., Science 2003;302:415-419.
72. Herzog R W. Gene therapy for scid-x1: Round 2. Molecular Therapy;18:1891.
73. Castro R J. Journal of Law and the Biosciences 2016;3:726-735.
74. Boussif et al., PNAS 1995;92:7297-7301.
75. Longo P A, et al., Methods in enzymology 2013;529:227-240.
76. Bartman et al., Exp Cell Res 2015;330:178-185.
77. Nagano et al., FEBS Lett 2002;524:111-115.
78. Hermann et al., Cell Stem Cell 2012;11:715-726.
79. Choi et al., Human Mutation 2010;31:788-793.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

1. A method of treating non-obstructive azoospermia (NOA) in a male subject caused by a genetic mutation, comprising:
introducing a recombinant nucleic acid molecule into spermatogonial stem cells (SSCs) from the testes of the male subject, wherein the nucleic acid molecule corrects the genetic mutation causing the NOA, thereby generating transformed SSCs;
isolating transformed SSCs that are heterozygous or hemizygous for the genetic mutation, thereby generating isolated transformed SSCs; and
introducing the isolated transformed SSCs that are heterozygous or hemizygous for the genetic mutation into the male subject, thereby treating NOA in the subject.

2. A method of treating non-obstructive azoospermia (NOA) in a male subject caused by a genetic mutation, comprising:
introducing a recombinant nucleic acid molecule into induced pluripotent stem cells (iPSCs) of the male subject, wherein the nucleic acid molecule corrects the genetic mutation causing the NOA, thereby generating transformed iPSCs;
isolating transformed iPSCs that are heterozygous or hemizygous for the genetic mutation, thereby generating isolated transformed iPSCs;
differentiating the isolated transformed iPSCs into primordial germ cell-like cells (PGCLCs); and
transplanting the PGCLCs into the testes of the male subject or differentiating the PGCLCs into sperm in vitro,
thereby treating NOA in the subject.

3. A method of treating premature ovarian insufficiency (POI) in a female subject caused by a genetic mutation, comprising:
introducing a recombinant nucleic acid molecule into induced pluripotent stem cells (iPSCs) of the female subject, wherein the nucleic acid molecule corrects the genetic mutation causing the POI, thereby generating transformed iPSCs;
isolating transformed iPSCs that are heterozygous or hemizygous for the genetic mutation, thereby generating isolated transformed iPSCs;
differentiating the isolated transformed iPSCs into primordial germ cell-like cells (PGCLCs); and
transplanting the PGCLCs into an ovary of the female subject or differentiating the PGCLCs into eggs in vitro,
thereby treating POI in the female subject.

4. The method of claim 1, further comprising:
prior to introducing the recombinant nucleic acid molecule into the isolated SSCs,
obtaining the SSCs from the testis of the male subject prior to introducing the recombinant nucleic acid molecule into the resulting isolated SSCs.

5. The method of claim 4, further comprising:
culturing ex vivo the isolated SSCs obtained from the testis prior to introducing the recombinant nucleic acid molecule.

6. The method of claim 1, further comprising:
culturing ex vivo the isolated transformed SSCs prior to introducing the transformed SSCs that are heterozygous or hemizygous for the genetic mutation into the male subject.

7. The method of claim 1, wherein isolating the transformed SSCs that are heterozygous or hemizygous for the genetic mutation comprises:
selecting individual transformed SSCs;
genotyping the individual transformed SSCs;
identifying individual transformed SSCs that are heterozygous or hemizygous for the genetic mutation; and
selecting the individual transformed SSCs that are heterozygous or hemizygous for the genetic mutation.

8. The method of claim 1, wherein the genetic defect causing the NOA or POI comprises a recessive mutation.

9. The method of claim 17, wherein the genetic defect causing the NOA or POI comprises a dominant mutation.

10. The method of claim 1, further comprising:
introducing sperm from the treated male subject into a female egg, thereby generating one or more embryos; and
selecting embryos that do not comprise the recombinant nucleic acid molecule,
wherein the recombinant nucleic acid molecule is not transmitted to progeny of the subject.

11. The method of claim 1, further comprising:
introducing sperm from the treated male subject into a female egg, thereby generating one or more embryos; and
selecting embryos that comprise the recombinant nucleic acid molecule,
wherein the recombinant nucleic acid molecule is transmitted to progeny of the subject.

12. The method of claim 3, further comprising:
fertilizing an egg from the treated female subject with sperm to produce one or more embryos.

13. The method of claim 12, wherein:
if the genetic defect causing the NOA or POI comprises a recessive mutation, the method further comprises selecting embryos that do not comprise the recombinant nucleic acid molecule, wherein the recombinant nucleic acid molecule is not transmitted to progeny of the subject, or
if the genetic defect causing the NOA or POI comprises a dominant mutation, the method further comprises selecting embryos that comprise the recombinant nucleic acid molecule, wherein the recombinant nucleic acid molecule is transmitted to progeny of the subject.

14. The method of claim 10, further comprising:
implanting the selected embryos into a uterus to establish a pregnancy.

15. The method of claim 1, further comprising:
collecting sperm from ejaculate, testis, or excurrent duct system of the testis of the treated male subject, or
obtaining eggs from the treated female subject.

16. The method of claim 1, wherein the genetic mutation causes another comorbid disease, and the method treats the comorbid disease in the treated subject and in progeny of the treated subject.

17.-20. (canceled)

21. The method of claim 1, wherein the recombinant nucleic acid molecule comprises a cDNA encoding a therapeutic gene.

22. The method of claim 1, wherein the recombinant nucleic acid molecule comprises a recombinant DNA template to direct homology directed modification of the subject's genome.

23. (canceled)

24. The method of claim 1, wherein the method further includes:
introducing a Cas9 protein or Cas9 encoding nucleic acid molecule into the SSCs from the testis of the male subject.

25. The method of claim 24, wherein the Cas9 protein and the recombinant nucleic acid molecule are complexed to one another, prior to introducing into SSCs from the testis of the male subject.

26. The method of claim 1, wherein the recombinant nucleic acid molecule targets an endogenous native locus associated with NOA, or targets or a safe harbor locus.

27.-28. (canceled)

29. The method of claim 1, wherein the recombinant nucleic acid molecule is introduced into the SSCs from the testis of the male subject using polyethyleneimine (PEI).

30. The method of claim 1, wherein the genetic mutation causing the NOA comprises a mutation in TEX11, GCNA, PORCN, MAGEB10, AKAP4, FMR1, SCML2, SOX3, MCM8, androgen receptor (AR), AFF4, AKAP9 or SOHLH1.

31. The method of claim 3, wherein the genetic mutation causing the POI comprises a mutation in MCM8, FMR1, or DCAF17.
